(19) United States
(12) Patent Application Publication
(10) Pub. No.: US 2007/0015965 A1
(43) Pub. Date: Jan. 18, 2007

(54) METHODS AND APPARATUS FOR COLONIC CLEANING

(75) Inventors: John A. Cox, Macungie, PA (US); Tracy Maahs, Rancho Santa Margarita, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 11/457,053

(22) Filed: Jul. 12, 2006

Related U.S. Application Data
(60) Provisional application No. 60/699,300, filed on Jul. 13, 2005.

(51) Int. Cl. A61B 1/00

(52) U.S. Cl. 600/114; 600/104; 600/156; 600/116; 600/144

(57) ABSTRACT

Apparatus and methods are provided for cleaning a patient's colon in preparation for colonoscopy. A rigidizable overtube is described, as well as several variations of a colon-cleaning element configured for use with the overtube to clean the patient's colon. In one variation, the colon-cleaning element comprises a fluid jet. In another variation, the colon-cleaning element comprises a mechanical brush. Aspiration optionally may be provided for removing blockage material disrupted during colonic cleaning.

### CROSS-REFERENCES TO RELATED APPLICATIONS

The present application claims the benefit of provisional U.S. Application No. 60/699,300 (Attorney Docket No. 021496-005000US), filed Jul. 13, 2005, the full disclosure of which is incorporated herein by reference.

### BACKGROUND OF THE INVENTION

### Field of the Invention

The present invention relates to apparatus and methods for preparing a patient for colonoscopy. More particularly, the present invention relates to methods and apparatus for cleaning the colon of a patient in preparation for colonoscopy.

The use of the colonoscope for examining the interior of the large intestine or colon is well-known. In general, a physician performing an examination or treatment of the colon inserts a colonoscope into the anus and then advances the colonoscope into the colon. A complete examination requires the physician to advance the colonoscope into the colon, negotiate the sigmoid colon, and left and right colic flexures up to the cecum. Advancement of the colonoscope is generally accomplished by manipulation of a steerable tip of the colonoscope, which is controlled at the proximal end of the device by the physician, in addition to torquing and pushing the scope forward or pulling it backward.

Problems regularly occur, however, when negotiating the colonoscope through the bends of the colon, such as at the sigmoid and left and right colic flexures. These problems arise because the colon is soft and has unpredictable fixation points to the viscera of the abdomen, and it is easily distensible. Consequently, after the steerable tip of the colonoscope is deflected to enter a new region of the colon, the principal direction of the force applied by the physician urging the proximal end of the device into the patient's colon is not in the direction of the steerable tip. Instead, the force is directed along the axis of the colonoscope towards the preceding bend(s), and causes yielding or displacement of the colon wall.

The loads imposed by the colonoscope on the colon wall can have a myriad of possible effects, ranging from patient discomfort to spastic cramp-like contractions of the colon and even possible perforation or dissection of the colon. Consequently, the colonoscope cannot be advanced as far as the cecum in up to one-sixth of all cases.

While previously-known apparatus and methods provide some suggestions for solving the difficulties encountered in advancing diagnostic or therapeutic instruments through easily distensible body organs, few devices are commercially available. Although the precise reasons for this lack of success are uncertain, previously-known devices appear to pose several problems.

In addition to difficulties encountered during advancement through the colon, in many cases, colonoscopy cannot be performed due to poor colon cleaning or preparation prior to the procedure. Procedures for patients with incomplete preparation are delayed and the patient usually must undergo another preparation regimen, typically involving ingestion of laxatives.

In view of the foregoing, it would be desirable to provide apparatus and methods for cleaning the colon prior to or during colonoscopy for patients presenting with an incomplete preparation.

### BRIEF SUMMARY OF THE INVENTION

Apparatus is provided comprising a proximal handle, an overtube coupled to the proximal handle and having a distal region, an atraumatic tip disposed on the distal region, and mechanisms for selectively locking the shape of the overtube to assist one or more diagnostic or therapeutic instruments to negotiate the tortuous or unsupported anatomy of a hollow body organ, rather than distending the wall of the organ. The apparatus includes a main lumen extending between the handle, overtube and atraumatic tip, through which a diagnostic or therapeutic instrument, such as an endoscope or colonoscope, may be translated.

In some variations, the apparatus further comprises a colon-cleaning element configured to clean the colon in preparation for colonoscopy.

In further aspects of the present invention, methods are provided for removing obstructing material from a colon, typically in conjunction with a colonoscopy or other screening procedure. The removal methods, however, could also be used with therapeutic protocols for removing problematic blockages from the colon and intestines.

In a first specific aspect, the methods comprise advancing an overtube into a patient's colon to a position proximal of the blockage. Usually, the overtube will be advanced in a flexible state and, once reaching the blockage, will be transitioned into a substantially rigid state to provide a pathway for advancing a colon-cleaning element through a central lumen thereof. The colon-cleaning element can then be used to disrupt the colonic blockage, typically by directing a jet of pressurized fluid from the element to the blockage. The pressurized fluid may be directed distally, proximally, or may be injected within the blockage in order to provide for the desired disruption. Optionally, the colon-cleaning element may further provide for mechanical disruption of the colonic blockage, for example using reciprocating bristles of a brush, rotating helical elements, providing for scraping, or the like. In all cases, the disrupted material will typically be aspirated from the colon, for example through a lumen of the overtube by applying a vacuum at a proximal location through the overtube. Alternatively or additionally, the disrupted blockage material may be aspirated through the colon-cleaning element itself.

All or a portion of the region being cleaned may be isolated in order to enhance removal of the disrupted material. For example, balloon or other element on the overtube and/or on the colon-cleaning element may be engaged against a wall of the colon in order to isolate the cleaning region on either side or both sides thereof.

Use of the overtube is advantageous since it permits exchange of the cleaning tool with a colonoscope or other diagnostic or treatment device. Using all these approaches, the colon may thus be cleaned in sequential fashion where the overtube is initially positioned (optionally being rigidized), a colon-cleaning element advance through a lumen of the overtube, and the colonic blockage disrupted as generally described above. After the blockage has been disrupted and removed, the overtube may be further advanced to bend or elsewhere in the colon, the overtube optionally rigidized, and a colonoscope or endoscope advance through the overtube to a position distal of the bend.

In a further aspect of the present invention, methods for clearing a colon comprise advancing a colon-cleaning element to a location approximate a blockage in the colon. A fluid from the element is directed into the blockage to disrupt the blockage, and the disrupted blockage material is then aspirated from the colon. The disrupted blockage material may be removed through the colon-cleaning element itself, or optionally may be removed through an overtube through which the colon-cleaning element has been introduced. In some instances, it may be advantageous to occlude or isolate a region around the blockage which is to be removed. For example, when using an overtube, an occlusion can be created at a distal end of the overtube to isolate a region distally of the overtube. Such isolation typically comprises expanding a balloon on the overtube. Alternatively, balloons or other isolating members can be provided on the colon-cleaning element as an alternative to or in addition to occlusion balloons on the overtube. By providing at least two isolating occluded balloons, the region surrounding a blockage may be fully isolated allowing removal of the disrupted blockage material with minimal loss into other regions of the colon.

### BRIEF DESCRIPTION OF THE DRAWINGS

Further features of the invention, its nature and various advantages will be more apparent from the accompanying drawings and the following detailed description of the preferred variations, in which:

FIG. 1 is a schematic view of a human colon illustrating a common difficulty encountered in advancing a colonoscope beyond the sigmoid colon;

FIG. 2 is a side view of illustrative apparatus of the present invention;

FIG. 3A is a side-sectional exploded view of nestable elements of a first variation of an overtube suitable for use in the apparatus of FIG. 2;

FIG. 3B is a side view of two of the nestable elements of FIG. 3A nested together;

FIG. 4 is a side-sectional view of a distal region of the apparatus of FIG. 2 constructed in accordance with principles of the present invention;

FIG. 5 is a side-sectional view of an illustrative arrangement of a mechanism suitable for use in the handle of the apparatus of FIG. 2;

FIG. 6 is a side-sectional view of the detail of a wire clamping system suitable for use in the handle of FIG. 5;

FIG. 7A-7C are schematic views of a method of using the apparatus of the present invention;

FIG. 8 is a schematic view of an alternative step in the method of using the apparatus of the present invention;

FIG. 9 is a side view of an alternative variation of the apparatus of the present invention;

FIGS. 10A-10C are schematic views of components of a tensioning mechanism suitable for rigidizing the overtube of the present invention, wherein the components provide a fail-safe mode;

FIG. 11 is a cut-away side view of a tensioning mechanism incorporating the components of FIGS. 10A and 10B within the handle of the apparatus of FIG. 2;

FIGS. 12A-12D are schematic perspective views of alternative components of a tensioning mechanism that each provide a fail-safe mode;

FIGS. 13A and 13B are, respectively, a side sectional view of a tensioning mechanism incorporating the pulley manifold of FIG. 12B within the handle of the apparatus of FIG. 2, and an indicator that displays the status of the overtube;

FIGS. 14A-14C are cut-away side views of an alternative tensioning mechanism that transitions the overtube of the present invention between flexible and rigid states with successive actuations;

FIG. 15 is a side sectional view of yet another alternative tensioning mechanism employing pneumatic actuation;

FIG. 16 is a side sectional view of a further alternative tensioning system that transitions the overtube of the present invention from a flexible state to a rigid state without substantial movement of a distal end of the overtube;

FIGS. 17A and 17B, respectively, are a side-section view of an alternative element suitable for use in the overtube of FIG. 2 and a roller element suitable for use with the element of FIG. 17A, respectively;

FIGS. 18A and 18B depict the use of lubricious rails in the overtube of the apparatus of FIG. 2 or 9 to facilitate passage of a diagnostic or therapeutic device through the main lumen;

FIG. 19 is a side-sectional view of an alternative nestable element having an integral lubricious lining;

FIGS. 20A and 20B are side-sectional views of alternative nestable elements that form a smooth internal lumen when nested together;

FIGS. 21A-21D are still further alternative variations of the nestable elements of FIG. 3, in which the nestable elements are macroscopically textured to enhance friction;

FIG. 22 is a schematic view of the lumen of the overtube of the present invention depicting the use of multiple devices;

FIGS. 23-28 depict side-sectional views of various alternative variations of an atraumatic tip constructed in accordance with the present invention;

FIGS. 29 and 30 are alternative variations of the overtube of the present invention, having tensioning systems that employ shape memory materials;

FIGS. 31A-31C are, respectively, a side-sectional view of an alternative variation of an overtube suitable for use in the present invention having a multiplicity of interconnected links surrounded by a clamping sleeve, and cross-sectional views of portions of the sleeve;

FIG. 32 is a side-sectional view of a further alternative variation of an overtube constructed in accordance with the present invention having a spiral bladder to actuate the clamping links;

FIG. 33 is a side-sectional view of another alternative variation of an overtube of the present invention having thermally-actuable bands;

FIGS. 34A and 34B are side-sectional views of a yet further alternative variation of an overtube of the present invention comprising a series of helical links having regions of different durometer;

FIG. 35 is a side-sectional view of a still further alternative variation of an overtube suitable for use with the present invention comprising a series of links having proximal and distal rims that interlock;

FIG. 36 is a side-sectional view of another alternative variation of the present invention comprising a series of links that form coacting joints;

FIG. 37 is a side-sectional view of yet another alternative variation of an overtube having thermally regulated stiffness;

FIGS. 38A-38C are schematic views of yet another alternative variation of an overtube suitable for use with the present invention, in which the diameters of tension wire lumens extending through the overtube vary responsive to electrical energization;

FIG. 39 is a side-sectional view of still another alternative variation of an overtube having a series of electrically activated links disposed in an overlapping fashion around a series of rigid links;

FIGS. 40A and 40B are side-sectional views of, respectively, an electrically activated nestable link and a plurality of the electrically activated nestable link of FIG. 40A nested together to form an overtube suitable for use with the apparatus of the present invention;

FIG. 41 is a side-sectional view of a disposable sheath for use with the overtube of the present invention;

FIG. 42 is a schematic side view of a strap that couples the apparatus of the present invention to a colonoscope;

FIGS. 43A-43C are, respectively, isometric, top and side views of a loading device for inserting a liner within apparatus of the present invention;

FIGS. 44A-44C are, respectively, isometric, top and side views of an alternative loading device, liner and apparatus handle of the present invention;

FIGS. 45A-45C are, respectively, isometric, top and side views of the apparatus of FIG. 44, illustrating a method of detaching the loading device from the liner;

FIGS. 46A and 46B are top and side views of the apparatus of FIGS. 44 and 45, illustrating a method of securing the liner to the apparatus handle; and

FIGS. 47A and 47B are top and side views of a variation of the apparatus of FIG. 46 comprising a fluid reservoir.

FIGS. 48A and 48B are schematic side views, partially in section, illustrating a variation of a colon-cleaning element and illustrating a method of cleaning the colon with the element.

FIG. 49 is a schematic side view, partially in section, illustrating a method of using a variation of the apparatus of FIG. 48 comprising an alternative colon-cleaning element.

FIG. 50 is a schematic side view, partially in section, illustrating a method of using another variation of the apparatus of FIG. 48 comprising another alternative colon-cleaning element.

FIG. 51 is a schematic side view, partially in section, illustrating a method of using another variation of the apparatus of FIG. 48 comprising yet another alternative colon-cleaning element.

FIG. 52 is a schematic side view, partially in section, illustrating a method of using a variation of the apparatus of FIG. 48 comprising a further alternative colon-cleaning element.

FIGS. 53A and 53B are a schematic side view, partially in section, and a side-sectional view, illustrating a method of using a variation of the apparatus of FIG. 48 comprising a still further alternative colon-cleaning element.

FIG. 54 is a schematic side view, partially in section, illustrating a method of cleaning the colon with a mechanical colon-cleaning element.

### DETAILED DESCRIPTION OF THE INVENTION

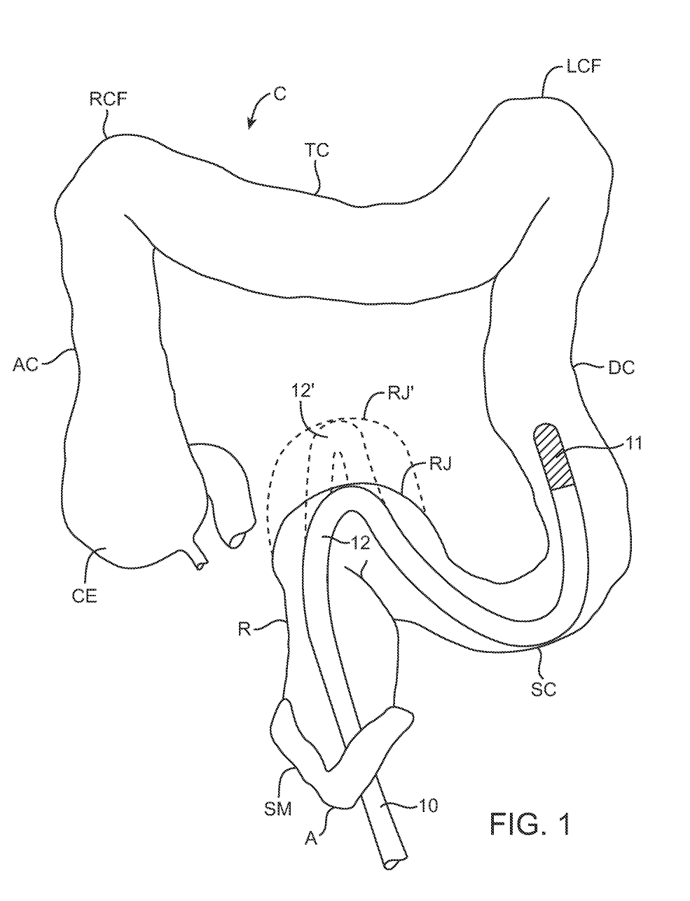

Referring to FIG. 1, problems associated with previously-known apparatus and methods for inserting and advancing a diagnostic or therapeutic instrument into a hollow body organ having tortuous or unsupported anatomy, illustratively, patient's colon C, are described. Colon C includes sphincter muscle SM disposed between anus A and rectum R. Rectum R is coupled via the rectosigmoid junction RJ to sigmoid colon SC. Sigmoid colon SC joins descending colon DC, which in turn is coupled to transverse colon TC via left colic flexure LCF. Transverse colon TC also is coupled by right colic flexure RCF to ascending colon AC and cecum CE, which receives waste products from the small intestine.

As illustrated in FIG. 1, colonoscope 10 having steerable distal tip 11 is typically inserted through anus A into rectum R, and then steered through rectosigmoid junction RJ into sigmoid colon SC. As depicted in FIG. 1, distal tip 11 of colonoscope 10 is advanced through sigmoid colon SC and deflected into descending colon DC. Further urging of the colonoscope by the physician can cause region 12 of the colonoscope to bear against and cause displacement of the rectosigmoid junction RJ, as illustrated by dotted lines 12′ and RJ′ in FIG. 1.

Such distension may result in patient discomfort or spasm, and if unnoticed, could result in injury to the colon. The potential for movement of colonoscope to cause distension, discomfort or spasm is also great where the colonoscope must negotiate left colic flexure LCF and right colic flexure RCF, and results in a large portion of such examinations terminating before the physician can advance distal tip 11 to cecum CE.

The present invention provides apparatus and methods for placing a diagnostic or therapeutic instrument through the tortuous or unpredictably supported anatomy of a hollow body organ, such as the esophagus or colon, while reducing the risk of distending or injuring the organ. Apparatus constructed in accordance with the present invention permits an endoscope or colonoscope to be readily advanced into a patient's tortuous or unsupported anatomy by selectively shape-fixing an overtube portion of the apparatus, while also preventing tissue from being captured or pinched between the overtube and scope.

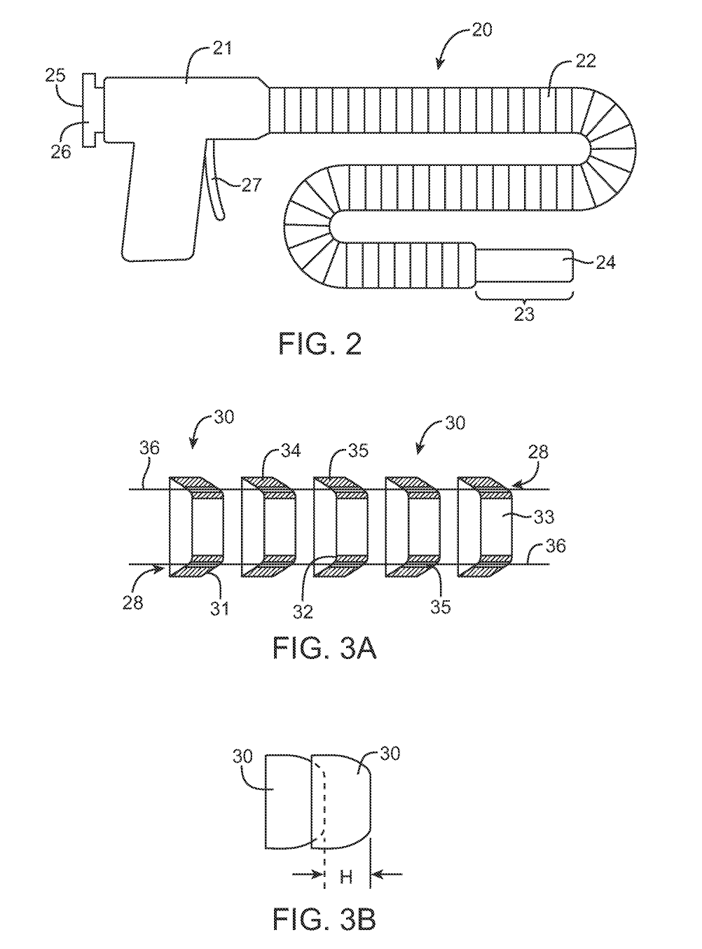

Referring now to FIG. 2, apparatus 20 of the present invention is described. Apparatus 20 comprises handle 21, overtube 22, and distal region 23 having atraumatic tip 24. Handle 21 includes lumen 25 that extends from Toughy-Borst valve 26 through overtube 22, distal region 23 and atraumatic tip 24. Lumen 25 is configured to facilitate passage of a standard commercially available colonoscope, such as colonoscope 10, therethrough. Toughy-Borst valve 26 may be actuated to releasably lock colonoscope 10 to apparatus 20 when colonoscope 10 is inserted within lumen 25. As described hereinafter, overtube 22 is configured so that it can be selectively transitioned between a flexible state and a rigid, shape-fixed state by actuator 27 disposed on handle 21.

In FIG. 3A, illustrative variation of overtube 22 comprises a multiplicity of nestable elements 30. For purposes of illustration, nestable elements 30 are shown spaced-apart, but it should be understood that elements 30 are disposed so that distal surface 31 of one element 30 coacts with proximal surface 32 of an adjacent element. Each of nestable elements 30 has central bore 33 to accommodate colonoscope 10, and preferably two or more tension wire bores 35. When assembled as shown in FIG. 2, nestable elements 30 are fastened with distal and proximal surfaces 31 and 32 disposed in a coacting fashion by a plurality of tension wires 36 that extend through tension wire lumens 28 defined by tension wire bores 35. Tension wires 36 preferably are made from a superelastic material, e.g., nickel titanium alloy, to provide flexibility, kink-resistance and smooth movement of the tension wires through tension wire bores 35. Alternatively, the tension wires may be made from braided stainless steel, a single stainless steel wire, Kevlar, a high tensile strength monofilament thread, or combinations thereof. These materials are provided only for the sake of illustration and should in no way be construed as limiting.

In a preferred variation, a ratio of the diameter of tension wires 36 to the diameter of tension wire bores 35 approximately is in a range of ½ to ⅔. Applicants have observed that this provides smooth relative movement between the tension wires and the nestable elements, even when overtube 22 is retroflexed. While a greater ratio is desirable, such a configuration appears to cause the edges of tension wire bores 35 to gouge into tension wires 36, thereby constraining movement of the tension wires through the tension wire bores. Conversely, while applicants contemplate that a smaller ratio would provide even smoother relative movement, the resultant increase in the thickness of wall 34 of each nestable element 30 is undesirable.

In a preferred variation, adjacent surfaces 31 and 32 of each nestable element 30 are contoured to mate with the next adjacent element, so that when tension wires 33 are relaxed, surfaces 31 and 32 can rotate relative to one another. Tension wires 36 are fixedly connected to the distal end of overtube 22 at the distal ends and to a tensioning mechanism disposed within handle 21 at the proximal ends. When actuated by actuator 27, tension wires 36 impose a load that clamps distal and proximal surfaces 31 and 32 of nestable elements 30 together at the current relative orientation, thereby fixing the shape of overtube 22.

When the load in tension wires 36 is released, tension wires 36 provides for relative angular movement between nestable elements 30. This in turn renders overtube 22 sufficiently flexible to negotiate a tortuous path through the colon. When the tensioning mechanism is actuated, however, tension wires 36 are retracted proximally to apply a clamping load to the nestable elements. This load prevents further relative movement between adjacent elements 30, and stiffens overtube 22 so that any distally directed force applied to colonoscope 10 causes distal tip 11 to advance further into the colon, rather than cause overtube 22 to bear against the wall of the colon. The shape-fixed overtube absorbs and distributes vector forces, shielding the colon wall.

In a preferred variation, the radius of curvature of proximal surface 32 closely approximates the radius of curvature of distal surface 31. In particular, a ratio of the radius of curvature of distal surface 31 to that of proximal surface 32 is in an approximate range of about 0.9 to 1.0. Furthermore, the coefficient of static friction between the distal and proximal surfaces preferably is in an approximate range of 0.2 to 1.4 (based on ASTM standard D1894). This structure appears to permit sufficient frictional force to develop between the surfaces to prevent relative movement between adjacent elements when overtube 22 is rigidized.

Nestable elements 30 may be configured to provide a stack-up of overtube 22 that is a function of the growth height. As defined in FIG. 3B, growth height H is the increase in the longitudinal length of overtube 22 when one nestable element 30 is nested within another nestable element 30. To accommodate the radius of curvature obtainable by standard commercially available colonoscopes, growth height H preferably is less than or equal to about 0.31 in, and more preferably about 0.16 in. This provides overtube 22 with sufficient flexibility to assume a radius of curvature that is less than or equal to approximately 0.95 in. If overtube 22 needs to accommodate other endoscopes or medical instruments that are larger or smaller in size and/or that may assume different radii of curvature, or the overtube needs to accommodate tighter anatomical constraints, applicants contemplate that growth height H may be increased or decreased proportionate to the change in dimensions of overtube 22. Applicants note that the preceding geometrical characterization of nestable element 30 does not account for material interference or effects to the tension wire bores, which are omitted from FIG. 3B for illustrative purposes.

Nestable elements 30 preferably are molded from a polymer filled with fibers of glass, carbon, or combinations thereof. In a particularly useful variation, nestable elements 30 are molded from polyurethane filled with 20-40% by volume of glass fibers, 20-40% by volume of carbon fibers, or 20-40% by volume of glass and carbon fibers. One example is isoplast 2540, which is available from Dow Chemicals, Midland, Mich. Applicants have observed that such materials enhance friction between adjacent elements, which advantageously reduces the risk of relative angular movement between the adjacent elements when overtube 22 is stiffened and, thus, reduces the risk of undesired reconfiguration of overtube 22 in its shape-locked state. While a greater amount of glass and/or carbon fibers is desirable, such a material appears to reduce the structural integrity of the nestable element.

Furthermore, fiber embedded polymers increase the rigidity of nestable elements 30 so that longitudinal contraction of overtube 22 is significantly reduced when the overtube is stiffened. Longitudinal contraction develops when tension wires 36 are actuated to apply a compressive clamping load to overtube 22. The resultant pressure eliminates any gaps between adjacent elements 30 and deflects the proximal portion of each nestable element radially outward. This foreshortens each element in the longitudinal direction, so that overtube 22 contracts in the axial length.

Typically, an overtube made from polymeric nestable elements without inclusion of glass and/or carbon fibers will contract approximately 8-12% in the longitudinal direction when a compressive force of approximately 30 lbs is applied. By comparison, when a compressive load of 30 lbs is applied to nestable elements made from a glass and/or carbon fiber embedded polymer, as in the preferred variation of the present invention, the overtube only contracts approximately 4%. Advantageously, this reduces trauma to the patient by providing greater accuracy during use of the present invention, which is particularly important in delicate procedures. In addition to glass and/or carbon filled polymers, it will be apparent to one of ordinary skill in the art that nestable elements 30 also may be molded or machined from other polymers and/or metals, such as polyurethane, polyvinyl chloride, polycarbonate, nylon, titanium, tungsten, stainless steel, aluminum, or combinations thereof. Indeed, nestable elements 30 made from metals experience a longitudinal contraction even less than that experienced by fiber embedded polymers. These materials are provided only for the sake of illustration, and one of ordinary skill in the art will recognize numerous additional materials that are suitable for use with the apparatus of the present invention.

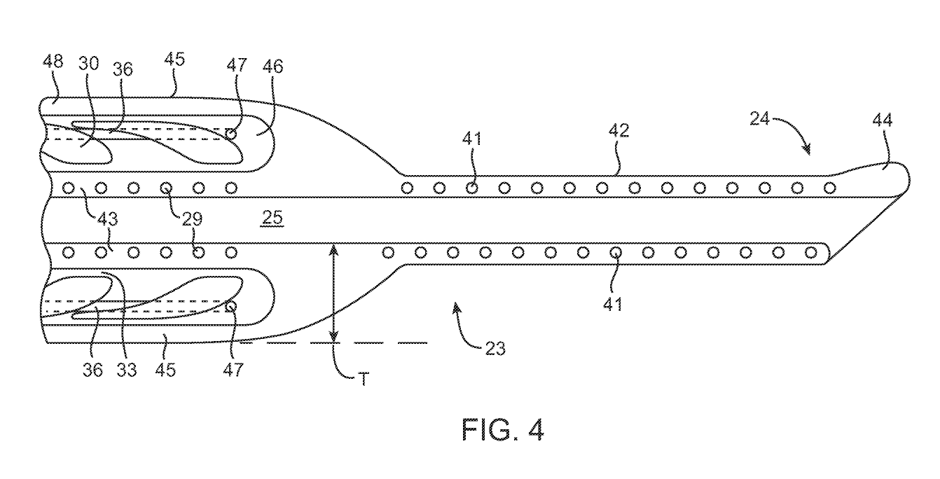

Referring now to FIG. 4, an illustrative variation of distal region 23 and atraumatic tip 24 is described. Distal region 23 comprises flexible, kink-resistant coil 41 encapsulated in flexible layer 42. Layer 42 preferably comprises a soft elastomeric and hydrophilic coated material, such as silicon or synthetic rubber, and terminates at the distal end in enlarged section 44 that forms atraumatic tip 24. At the proximal end, layer 42 joins with or is integrally formed with liner 43 that extends through bores 33 of nestable elements 30 to handle 21. In a preferred variation, liner 43 is made of a thin, flexible material optionally having flexible, kink-resistant coil 29 embedded therein. The material of liner 43 preferably has a high durometer within the range of 30-80 D, but also may have a lower or higher durometer.

Layer 42 preferably joins with or is integrally formed with flexible elastomeric skin 45 to form sheath 48, which encapsulates nestable elements 30 in annular chamber 46. Skin 45 provides a relatively smooth outer surface for overtube 22, and prevents tissue from being captured or pinched during relative rotation of adjacent nestable elements 30. In a preferred variation, aggregate thickness T of skin 45, nestable elements 30 and liner 43, is less than or equal to approximately 2.5 mm, and more preferably less than or equal to 1 mm. For example, skin 45 may have a thickness of 0.13 mm, or more preferably 0.1 mm, element 30 may have a thickness of 1.9 mm, and more preferably 0.7 mm, and liner 43 may have a thickness of 0.38 in, or more preferably 0.15 mm.

In accordance with one aspect of the present invention, colonoscope 10 may be positioned with its distal tip 11 disposed in distal region 23, so that deflection of steerable distal tip 11 imparts an angular deflection to distal region 23 and atraumatic tip 24. To ensure that there is no gross relative motion between colonoscope 10 and apparatus 20, Toughy-Borst valve 26 is tightened to engage apparatus 20 to the colonoscope. In this manner, colonoscope 10 and distal region 23 may be simultaneously advanced through the colon, with the distal tip of the colonoscope providing a steering capability to apparatus 20. Apparatus 20 therefore may be advantageously advanced together with colonoscope 10 when overtube 22 is in the flexible state, reducing relative motion between apparatus 20 and colonoscope 10 to those instances where overtube 22 must be shape-locked to prevent distension of the colon.

Still referring to FIG. 4, terminations 47 of tension wires are described. Terminations 47 illustratively comprise balls welded or molded onto the ends of tension wires 36 that ensure the tension wires cannot be pulled through tension wire bores 35 of the distal-most nestable element 30. This ensures that the nestable elements cannot come loose when overtube 22 is disposed within a patient.

Alternatively, terminations 47 may comprise knots formed in the ends of tension wires 36, or any suitable fastener that prevents the tension wires from being drawn through the tension wire bores of the distal-most nestable element. Advantageously, skin 45 provides additional assurance that all of nestable elements 30 can be safely retrieved from a patient's colon in the unlikely event of a tension wire failure.

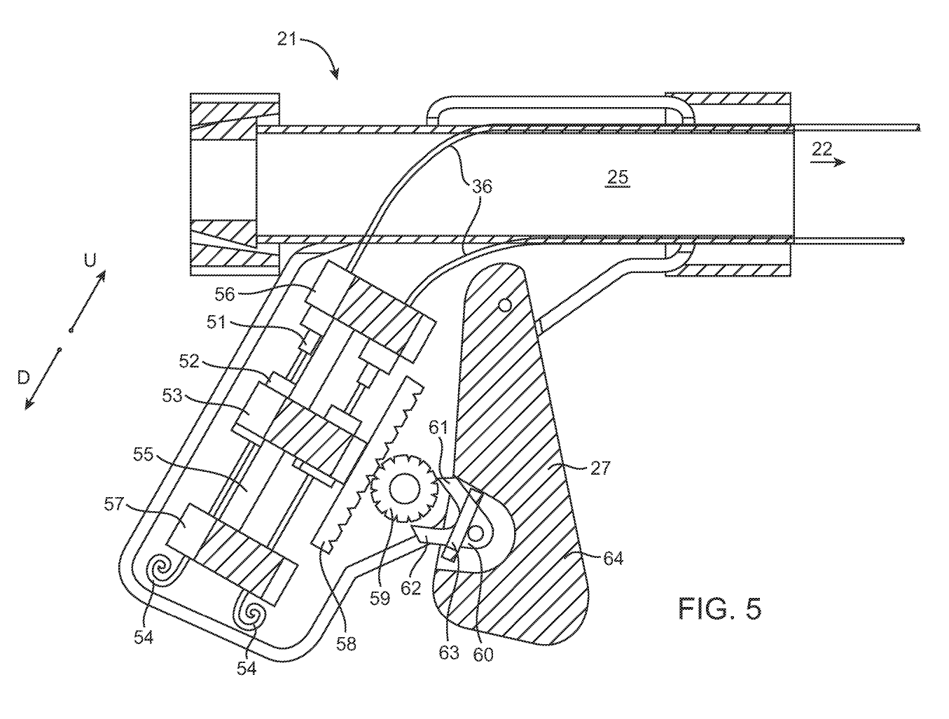

Referring now to FIGS. 2 and 5, tension wires 36 within overtube 22, liner 43 and lumen 25 extend from distal region 23, through overtube 22, and to handle 21. Within handle 21, each tension wire 36 passes through wire lock release 51 fixedly attached to handle 21, and wire lock 52 disposed on slide block 53. Each tension wire 36 terminates at wire tension spring 54, which maintains tension wires 36 in light tension even when overtube 22 is in the flexible state. The degree of tension provided by wire tension springs 54 is not sufficient to clamp adjacent nestable elements 30 together, but on the other hand does not let gaps form between adjacent nestable elements, and helps to manage the tension wire take up or slack as overtube 22 makes various bends.

Slide block 53 is keyed to slide along rail 55 disposed between limit blocks 56 and 57, and comprises a rigid block having a bore through which rail 55 extends and an additional number of bores as required for the number of tension wires 36 employed. Rack gear 58 is fixedly coupled to slide block 53. Rack 58 mates with pinion gear 59, which is in turn driven by bi-directional pawl 60 coupled to actuator 27. Pinion gear 59 may be selectively engaged by either prong 61 or 62 of bi-directional pawl 60, depending upon the position of selector switch 63.

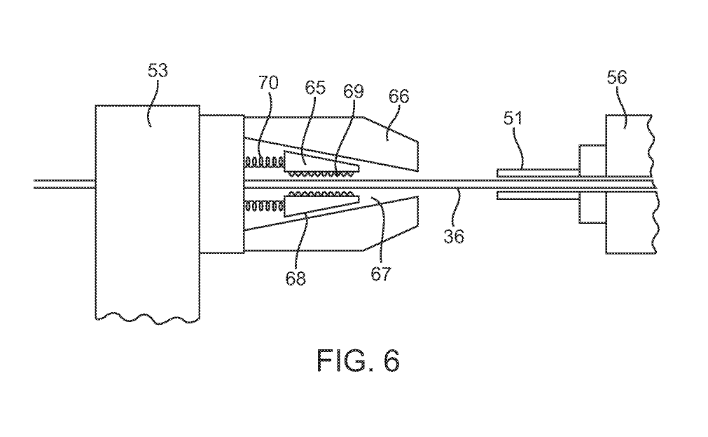

If prong 61 is selected to be engaged with pinion gear 59, a squeezing action applied to actuator 27, illustratively hand grip 64, causes rack 53 to move in the D direction in FIG. 5, thereby applying tension to tension wires 36. Repeated actuation of hand grip 64 causes slide block 53 to move progressively further in direction D, thereby applying an increasing clamping load on nestable elements 30. Any slack lengths of tension wires 36 extending below slide block 53 are taken up by wire tension springs 54. As discussed in greater detail below with respect to FIG. 6, wire locks 52, which are affixed to slide block 53, engage and retract tension wires 36 concurrently with movement of slide block 53 in the D direction.

If prong 62 is instead chosen by selector switch 63 to engage pinion gear 59, repeated actuation of hand grip 64 causes slide block 53 to translate in direction U, thereby relaxing the tensile load applied by tension wires 36 to nestable elements 30. Repeated actuation of hand grip 64 causes slide block 53 to advance in direction U until wire lock releases 51 engage wire locks 52, releasing all tension from tension wires 36 except that provided by wire tension springs 54. This action permits the clamping forces imposed on nestable elements 30 to be progressively reduced and render overtube 22 progressively move flexible, until when wire lock releases 51 engage wire locks 52, the overtube is returned to its most flexible state.

Referring to FIG. 6, wire lock 52 and lock release 51 are described in greater detail. Wire lock 52 includes jaws 65 disposed within collet 66. Collet 66 includes a tapered conical bore 67. Jaws 65 have ramped exterior surfaces 68 and teeth 69, and are biased against the surface formed by the tapered conical bore by springs 70. Teeth 69 are configured to engage tension wire 36 under the bias force of springs 70. When slide block 53 is moved in direction D (see FIG. 5), jaws 65 engage and grasp tension wire 36 and retract the tension wire in direction D.

To disengage teeth 69 from tension wire 36, e.g., when it is desired to allow overtube 22 to return to a flexible state, slide block 53 is actuated as described previously to move in direction U. Further actuation of slide block 53 towards limit block 56 and wire lock release 51 causes wire lock release 51 to extend into tapered conical bore 67 and push jaws 65 backward against the bias of springs 70. Once tension wires 36 are freed from jaws 65, overtube 22 returns to its most flexible state.

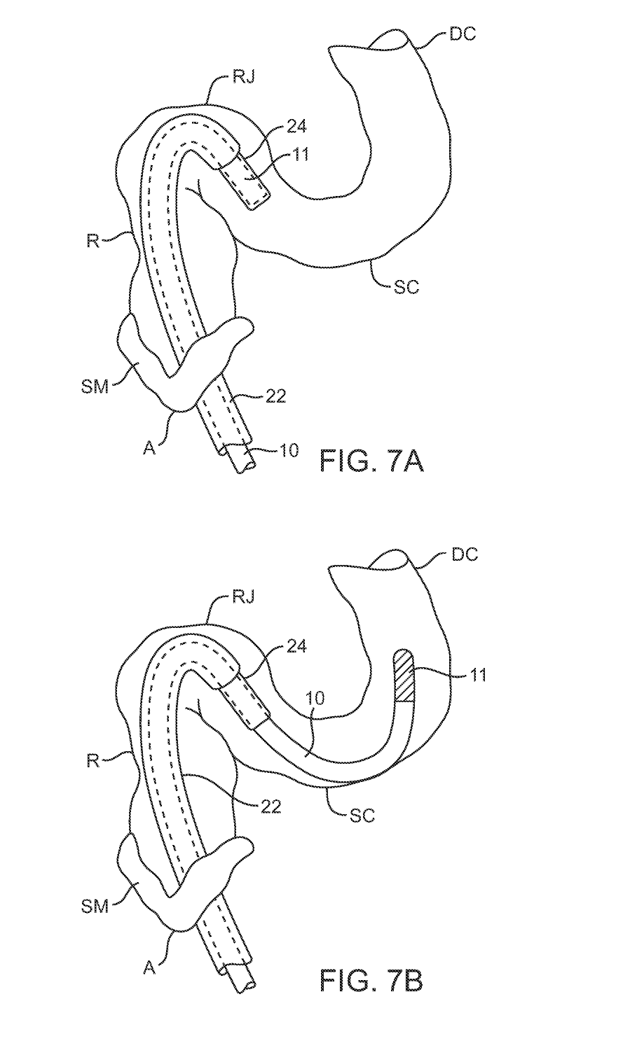

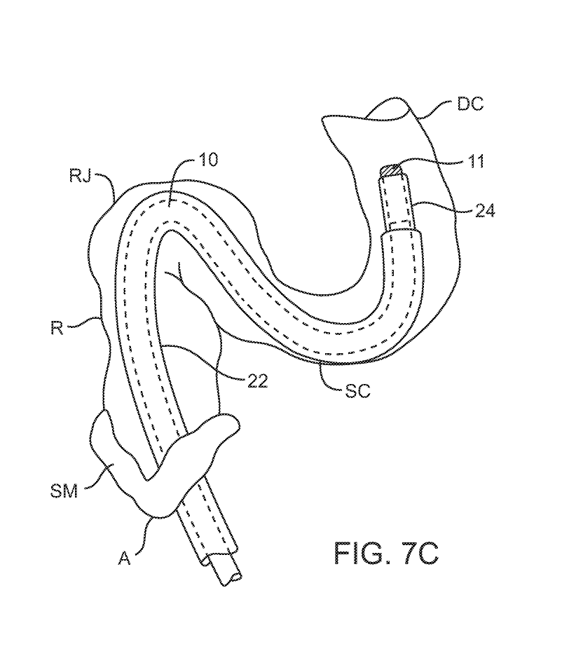

Referring to FIGS. 7A-7C, a method of using apparatus 20 is described. Colonoscope 10 and overtube 22 may be inserted into the patient either simultaneously or by first backloading the overtube onto the colonoscope. To perform simultaneous insertion, colonoscope 10 is introduced into lumen 25 of handle 21 until distal tip 11 of the colonoscope is disposed in distal region 23. Toughy-Borst valve 26 is actuated to lock apparatus 20 to colonoscope 10. As one unit, colonoscope 10 and overtube 22 are inserted into rectum R of the patient, and navigated about rectosigmoid junction RJ. As discussed previously, steerable distal tip 11 may be used to impart angular deflection to flexible tip 24 to steer tip 24 about tortuous curves, such as rectosigmoid junction RJ. Once distal tip 11 and tip 24 have been negotiated past rectosigmoid junction RJ, the current shape of overtube 22 is locked in the manner discussed above to provide a rigid channel through which colonoscope 10 may be further advanced into the colon without distending rectosigmoid junction RJ. Once distal tip 11 of colonoscope 10 is negotiated past sigmoid colon SC, overtube 22 is released from its rigid state and advanced along colonoscope 10 until it too traverses sigmoid colon SC. Again, the current shape of overtube 22 is locked to provide a rigid channel for advancement of colonoscope 10. To negotiate the remainder of the colon, such as left colic flexure LCF and right colic flexure RCF, the preceding steps may be repeated. In this manner, colonoscope 10 and overtube 22 may be navigated through the tortuous curves of the colon without distending the colon, and thereby causing discomfort, spasm or injury.

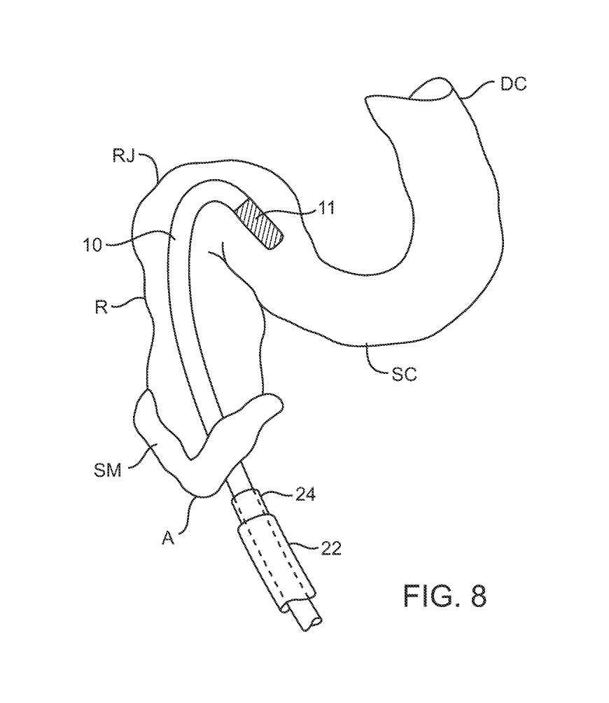

Alternatively, rather than simultaneously inserting both colonoscope 10 and overtube 22 into the patient, apparatus 20 first may be backloaded onto the colonoscope. First, overtube 22 is threaded onto colonoscope 10 and positioned proximal distal tip 11, as shown in FIG. 8. Colonoscope 10 then is inserted into rectum R of the patient and advanced around rectosigmoid junction RJ. Overtube 22 is advanced along colonoscope 10 into rectum R of the patient, using colonoscope 10 as a guide rail to negotiate rectosigmoid junction RJ. Once overtube 22 traverses rectosigmoid junction RJ to the position shown in FIG. 7A, the shape of overtube 22 is locked to provide a rigid channel through which colonoscope 10 may be further advanced into the colon. To negotiate the remainder of the colon, the steps discussed in reference to FIGS. 7B-7C may be performed.

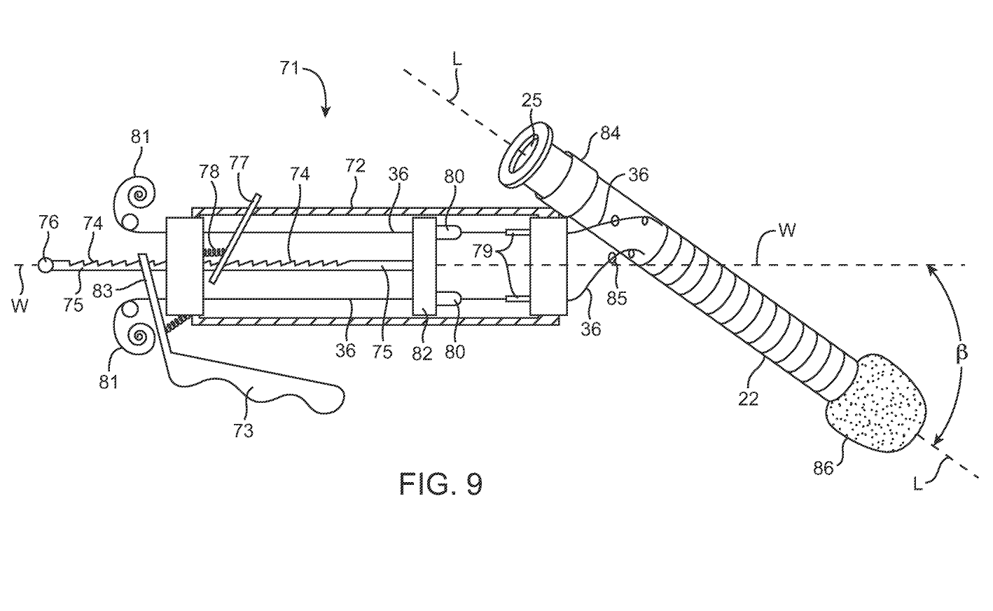

With respect to FIG. 9, an alternative variation of handle 21 is described. Like handle 21 of FIG. 5, handle 71 also embodies a ratchet-type tension mechanism, but in this variation overtube 22 may be separated from handle 71, thereby permitting handle 71 to be sterilized for repeated use. Handle 71 comprises housing 72 having actuator 73 that engages teeth 74 disposed along the length of rod 75, which defines working axis W of handle 71. Push knob 76 is affixed to the proximal end of rod 75 so that when pawl 77 is released, rod 75 may be pushed in a distal direction. Pawl 77 engages teeth 74 of rod 75 to prevent distally-directed motion of rod 75. Spring 78 biases pawl 77 against teeth 74 of rod 75, to provide a one-way ratchet effect when actuator 73 is squeezed.

As in the variation of FIG. 5, tension wires 36 extend through wire lock releases 79, wire locks 80, and are coupled to wire tension springs 81. Wire locks 80 are affixed to block 82, which translates within housing 72 responsive to movement of rod 75. Wire locks 80 and wire lock releases 79 operate in the same manner as described with reference to FIG. 6.

In operation, squeezing actuator 73, illustratively a hand grip, causes fork 83 to move rod 75 in a proximal direction so that pawl 77 captures the next distal-most tooth 74. This movement also causes wire locks 80 to engage and grasp tension wires 36 and retract the tension wires proximally. Further actuation of actuator 73 causes overtube 22 to stiffen in the manner previously described. Spring 78 retains pawl 77 in continuous engagement with teeth 74, thereby preventing rod 75 from moving in the distal direction.

When it is desired to make overtube 22 more flexible, pawl 77 is released and knob 76 pushed in the distal direction so that wire locks 80 engage wire lock releases 79. As described above, this releases tension wires 36 from wire locks 80 and permits overtube to assume its most flexible state.

In accordance with one aspect of the present invention, overtube 22 of the variation of FIG. 9 may be replaceably removed from yoke 84 of handle 71. In addition tension wires 36 further may comprise connectors 85 that permit the tension wires to be disconnected. Such a configuration permits the overtube to be removed and discarded after a single use, while the handle may be sterilized and reused.

Yoke 84 is also configured to position overtube 22 so that longitudinal axis L of the overtube is angularly displaced from working axis W by a predetermined angle β. This arrangement prevents handle 71 from interfering with advancement of colonoscope 10 into lumen 25.

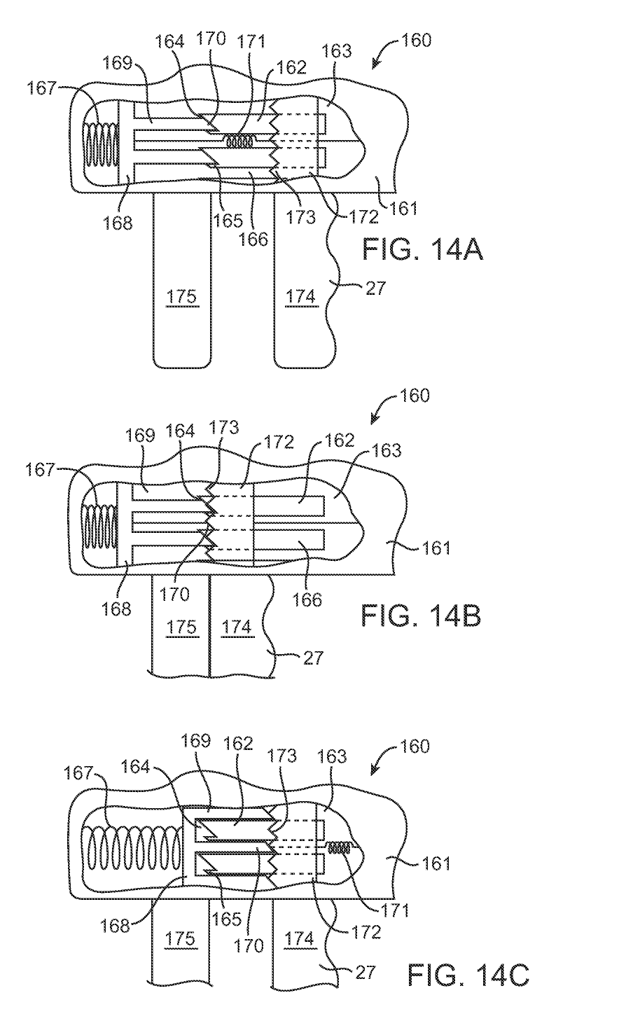

In accordance with yet another aspect of the present invention, overtube 22 includes atraumatic tip 86 that comprises a soft foam-like material. Atraumatic tip 86 not only facilitates advancement of overtube 22 in traversing tortuous anatomy, but also serves to retain the organ wall a safe distance away from the opening through which the colonoscope is reciprocated by radially expanding the organ wall in the vicinity of the tip, as described hereinbelow with respect to FIG. 14A. Accordingly, atraumatic tip 86 reduces the potential for tissue to be caught or pinched in lumen 25 when the colonoscope is manipulated.

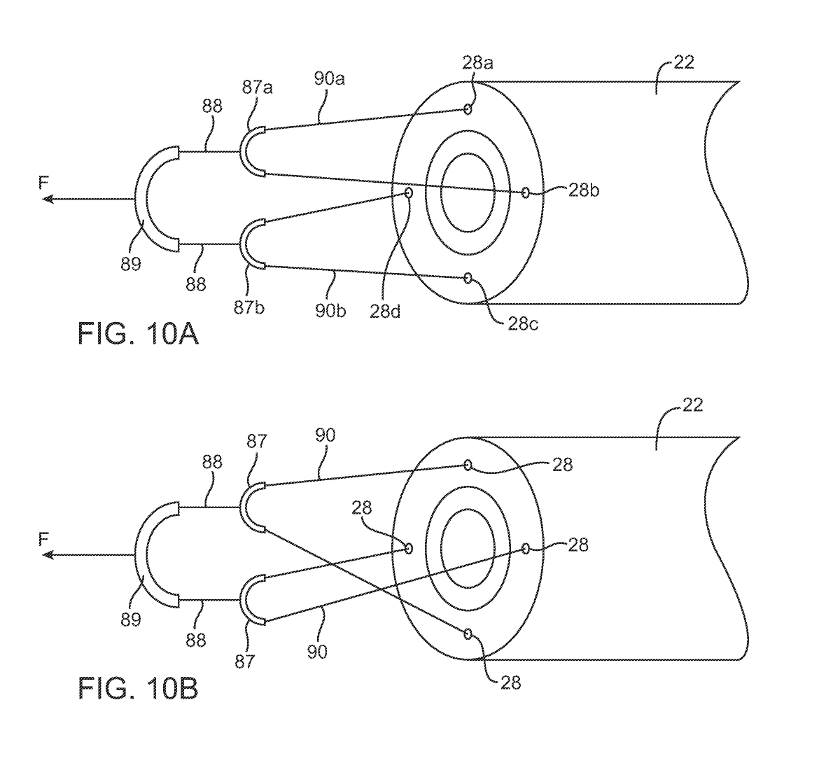

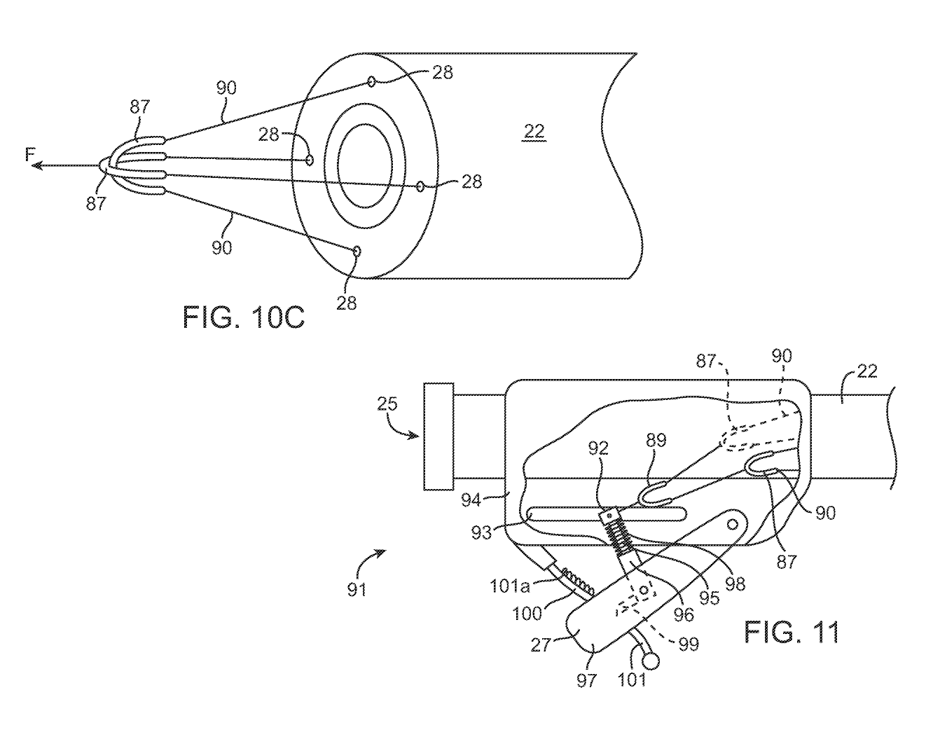

Referring now to FIGS. 10-16, alternative tensioning mechanisms are described, in which the tensioning mechanisms may provide a fail-safe mode that reduces the risk of undesired reconfiguration of the overtube in the event of tensioning mechanism failure. When overtube 22 is in the rigid state, the following tensioning mechanisms are configured to self-equalize compressive loads applied to the multiplicity of nestable elements, so that if, e.g., a tension wire breaks, the overtube either softens into the flexible state or retains its shape-locked state.

FIG. 10A schematically depicts components of a first variation of an alternative tensioning mechanism having plurality of distal pulleys 87 operably coupled via proximal tension wire 88. Proximal tension wire 88 is slidably disposed within proximal pulley 89. Each tension wire 90 couples adjacent tension wire lumens 28, through respective distal pulleys 87. For example, if four tension wire lumens 28a-28d are provided, as in FIG. 10A, first tension wire 90a extends from tension wire lumen 28a to adjacent tension wire lumen 28b through first distal pulley 87a. Likewise, second tension wire 90b extends from tension wire lumen 28c to adjacent tension wire lumen 28d through second distal pulley 87b.

This configuration equalizes tension within tension wires 90, so that a proximally directed force F applied to proximal pulley 89 is distributed evenly through tension wires 90. When one of the tension wires breaks, this configuration allows overtube 22 to soften into its flexible state since the loss of tension in any of the tension wires is transmitted through the pulley system to the remaining tension wires.

It will be apparent to one of ordinary skill in the art that tension wires 90a and 90b may comprise either two separate lengths of wire, or a single length of wire that is looped backwards after traversing the distal-most nestable element 30. Furthermore, while FIG. 10A depicts tension wires 90 extending through adjacent tension wire lumens 28, the tension wires instead may extend through wire lumens disposed diametrically opposite each other, as shown in FIG. 10B. Tension wires 90 preferably are made from a superelastic material, e.g., nickel titanium alloy, but also may be made from braided stainless steel, single stainless steel wires, Kevlar, a high tensile strength monofilament thread, or combinations thereof. These materials are provided only for the sake of illustration and should in no way be construed as limiting.

In an alternative variation illustrated in FIG. 10C, proximal pulley 89 is eliminated, and distal pulleys 87 are fixed to each other, e.g., by welding, so that a unitary pulley manifold is formed. A proximally directed force F that is applied to the pulley manifold is distributed evenly through tension wires 90 that extend through respective distal pulleys 87 to diametrically disposed tension wire lumens 28 within overtube 22. If tension wires 90 comprise two separate lengths of wires, the risk of reconfiguration of overtube 22 is reduced if one of the wires breaks since the tension within the overtube, as defined by the unbroken tension wire, is symmetrically balanced. If the remaining tension wire breaks, the tension wire relaxes into the flexible state. If tension wires 90 comprise a single length of wire that breaks, the overtube immediately relaxes into the flexible state, thereby also reducing the risk of undesired configuration of the overtube in the event of tensioning system failure.

Furthermore, applicants have observed that the apparatus of the present invention also may comprise only one distal pulley 87 coupled to overtube 22 via a single tension wire 90 disposed through diametrically opposite tension wire lumens 28. When a proximally directed force is applied to the single distal pulley, the force is distributed through the single tension wire to impose a symmetrical compressive clamping load on overtube 22 that is sufficient to shape-lock the overtube. When tension wire 90 breaks, overtube 22 immediately softens into its flexible state, thereby reducing the risk of undesired reconfiguration of the overtube in the event of tensioning system failure.

Referring now to FIG. 11, lumen 25 and tension wires 90 within overtube 22 extend from the distal region of the apparatus, through overtube 22, and to handle 91. Within handle 91, the tension wires are slidably coupled to distal pulleys 87, which in turn are slidably coupled to proximal pulley 89. Proximal pulley 89 is coupled to and translates with slide block 92, that is keyed to travel along track 93 disposed within housing 94. Plunger 95 is mounted pivotally to slide block 92 at the proximal end and slidably disposed within plunger housing at a distal end.

Plunger housing 96 is mounted pivotally to actuator 27, illustratively hand grip 97. To bias hand grip 97 against actuation absent an externally applied force, compression spring 98 is provided concentrically disposed about plunger 95. Compression spring 98 maintains tension wires 90 in constant tension when the tensioning mechanism is actuated to impose a clamping load. Advantageously, if adjacent nestable elements shift slightly when overtube 22 is shape-locked, the proximal bias of compression spring 98 immediately advances slide block 92 in the proximal direction to maintain a relatively constant tension load within tension wires 90, thereby reducing the risk of reconfiguration of the overtube back to the flexible state that otherwise may occur absent compression spring 98.

Hand grip 27 also includes pawl 99, which is disposed to engage teeth 100 on ratchet bar 101 to prevent distally-directed motion of slide block 92. Ratchet bar 101 is pivotally mounted in housing 94 with a spring (not shown) that, with the aid of compression spring 98, biases pawl 99 against teeth 100 of ratchet bar 101, to provide a one-way ratchet effect when hand grip 97 is squeezed.

In operation, squeezing hand grip 97 causes pawl 99 to capture the next proximal-most tooth 100. This movement also provides a compressive force to compression spring 98 that is transmitted to slide block 92. The proximally-directed component of the compressive force causes slide block 92 to translate along track 93, proximally retracting tension wires 90 so that a clamping load is imposed on the nestable elements within overtube 22. Further actuation of hand grip 97 causes overtube 22 to stiffen progressively in the manner previously described.

Advantageously, proximal-most tooth 100a is disposed on ratchet bar 101 at a predetermined proximal location that permits a single actuation of hand grip 97 to completely transition overtube 22 from its flexible state to its shape-fixed state. Furthermore, as pawl 99 advances hand grip 97 closer to housing 94, the mechanical advantage of the actuation of the hand grip increases. More specifically, as hand grip 97 becomes increasingly horizontal, the proximally-directed component of the force transmitted by compression spring 98 increases in magnitude. Accordingly, more force is transmitted to increase tension within tension wires 90, and thus increase the clamping load applied to rigidize overtube 22.

When it is desired to transition overtube 22 into the flexible state, pawl 99 is released from engagement with teeth 100 by rotating ratchet bar 101 in the proximal direction. The release of the compressive load applied to compression spring 98 causes hand grip 97 to rotate in the distal direction and slide block 92 to retract in the distal direction. This sufficiently relaxes tension wires 90 so that the tension wires retain little to no tension, thereby permitting overtube 22 to assume its most flexible state.

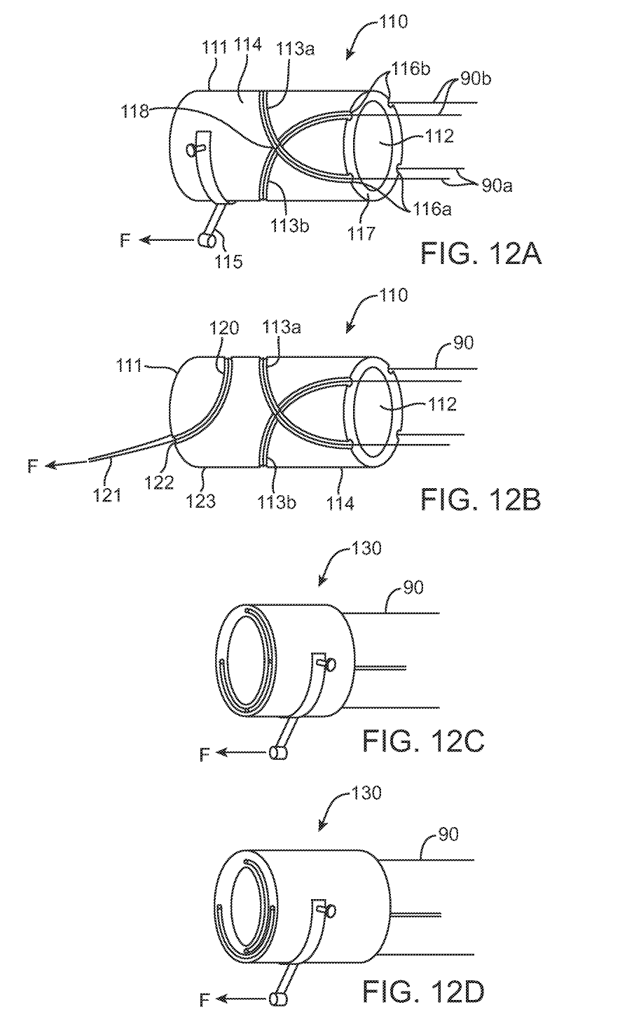

Referring now to FIGS. 12A-12D, alternative variations of fail-safe tensioning mechanisms are described, in which the plurality of pulleys of the previous variation is replaced by a single pulley manifold. In FIG. 12A, a first variation of a pulley manifold is described. Pulley manifold 110 includes body 111 having central bore 112 that accommodates colonoscope 10, first and second grooves 113a and 113b that each accept a tension wire, and are milled or molded into lateral surface 114 of body 111, and yoke 115 that is configured to couple pulley manifold 110 to an actuator (not shown).

First groove 113 includes a curved track that terminates at first distal ends 116a disposed diametrically opposite each other at distal surface 117. Second groove 113b also comprises a curved track that crosses first groove 113a at intersection 118, and terminates at second distal ends 116b. Second distal ends 116b are disposed at distal surface 117 diametrically opposite each other and preferably 45o from first distal ends 116a. Similar to distal pulleys 87 of FIG. 10B, each groove accepts a tension wire that extends through diametrically disposed tension wire lumens within overtube 22. To reduce friction between tension wires 90a and 90b at intersection 118, first groove 113a may have a greater depth than that of second groove 113b, or vice versa. To prevent tension wires 90 from disengaging from grooves 113, a sleeve (not shown) may be disposed around pulley manifold 110.

If tension wires 90 comprise two separate lengths of wires, the risk of reconfiguration of overtube 22 is reduced if one of the wires breaks since the tension within the overtube, as defined by the unbroken tension wire, is symmetrically balanced. If the remaining tension wire breaks, the overtube relaxes into the flexible state. If tension wires 90 comprise a single length of wire, the overtube immediately relaxes into the flexible state if the single wire breaks. Accordingly, pulley manifold 110 provides overtube 22 with a fail-safe mode that reduces the risk of reconfiguration of the overtube in the event of tensioning mechanism failure.

FIG. 12B depicts pulley manifold 110, in which the yoke is replaced with third groove 120. Third groove 120 is milled or molded into lateral surface 114, and accepts an additional tension wire 121 that may be coupled to actuator 27 (see FIG. 2). When a proximally directed force F is applied to tension wire 121, the force imposes tension to tension wires 90. Third groove 120 includes a curved track that terminates at third distal ends 122, which preferably are diametrically disposed opposite each other at proximal surface 123 of pulley manifold 110.

With respect to FIGS. 12A and 12B, an alternative variation of a pulley manifold is described. Rather than having grooves disposed on a lateral surface of the pulley manifold, pulley manifold 130 incorporates first and second grooves 131a and 131b that terminate at tension wire bores 132 disposed through body 133. Preferably, tension wire bores 132 are equidistantly and circumferentially disposed on proximal surface 136. Pulley manifold 130 also incorporates central bore 134 that accommodates colonoscope 10, and yoke 135 that couples pulley manifold 130 to actuator 27 (see FIG. 2).

In FIG. 12C, first and second grooves 131a and 131b are milled or molded in overlapping fashion. To reduce friction between tension wires disposed within the overlapping portion of the grooves, first groove 131a may have a depth greater than that of second groove 131b, or vice versa. In FIG. 12D, the first and second grooves do not overlap, first groove 131a having a smaller radius of curvature than that of second groove 131b.

Applicants also contemplate that either the first or second groove of the pulley manifolds of FIGS. 12A-12D may be eliminated so that a proximal force F applied thereto would impose a symmetrical compressive clamping force to overtube 22 through a single length of tension wire 90 that extends through diametrically disposed tension wire lumens. Accordingly, when tension wire 90 or 121 breaks, or yoke 115 fails, the overtube relaxes back into its flexible state, thereby reducing the risk of undesired reconfiguration of the overtube.

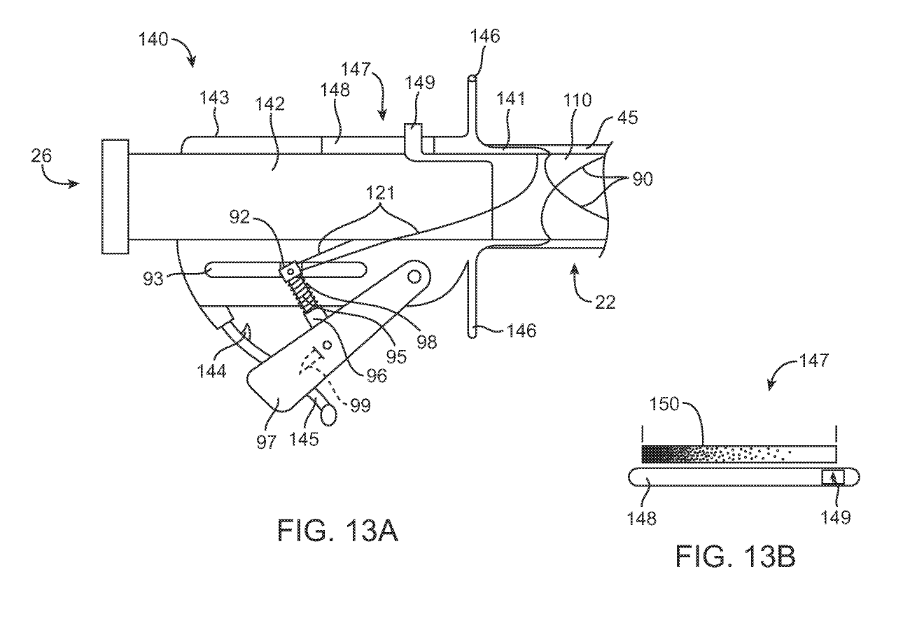

Referring now to FIGS. 13A and 13B, handle 140 is described employing pulley manifold 110 of FIG. 12B. Tension wires 90 within overtube 22, skin 45, liner (not shown for illustrative purposes) and lumen 25 extend from distal region 23 (see FIG. 2), through overtube 22, and to handle 140, which preferably measures less than or equal to 5 inches, similar to the other handle variations described herein. Within handle 140, tension wires are slidably coupled to pulley manifold 110, which rides within cylindrical extension 141 and cylinder 142. Cylindrical extension 141 may be integrally manufactured with housing 143, and is configured to be inserted into a patient's rectum. Concentric with cylindrical extension 141, cylinder 142 defines the proximal portion of lumen 25 disposed within handle 140.

Via additional tension wire 121, pulley manifold 110 is coupled to slide block 92, which is keyed to translate in track 93. As in handle 91 of FIG. 11, plunger 95 is coupled pivotally to slide block 92 at a proximal end, and slidably disposed within plunger housing 96 at a distal end. Concentrically disposed about plunger 95, compression spring 98 biases hand grip 97 from being actuated absent an externally applied force. As in FIG. 11, compression spring 98 maintains the level of tension within tension wires 90 if adjacent nestable elements shift slightly when overtube 22 is in the rigid state, thereby reducing the risk of reconfiguration of the overtube back to the flexible state.

Hand grip 97 also includes pawl 99, which is configured to engage tooth 144 of ratchet bar 145 to prevent distally-directed motion of slide-block 92. Tooth 144 is disposed on ratchet bar 145 at a predetermined proximal location that permits a single actuation of hand grip 97 to completely transition overtube 22 from its flexible state to its shape-fixed state. Ratchet bar 145 is mounted pivotally in housing 143 with a spring (not shown) that, with the aid of compression spring 98, biases pawl 99 against tooth 144. To release tension from tension wires 90, pawl 99 may be released from engagement with tooth 144 by rotating ratchet bar 145 in the proximal direction. This sufficiently relaxes tension wires 90 so that the tension wires retain little to no tension, thereby permitting overtube 22 to assume its most flexible state.

Handle 140 also has shield 146 coupled to a distal end thereof. Shield 146 prevents handle 140 proximal thereto from inadvertently being inserted into the patient's rectum. Handle 140 also incorporates indicator 147 (FIG. 13B) that provides a clinician with information about the rigidity of overtube 22. Indicator 147 comprises slot 148 disposed through a wall of housing 143, pointer 149 disposed through slot 148, and scale 150 disposed on an external surface of housing 143 adjacent to slot 148. Pointer 149 is coupled to translation of proximal manifold 110 so that it translates with the manifold. Scale 150 incorporates color gradations, or indicia (not shown) to indicate the rigidity of overtube 22. Of course, it will be obvious to one of ordinary skill in the art that pointer 149 may be coupled to any structure within handle 140 that moves when actuator 27 is actuated, e.g., slide block 92 or pawl 99. Alternatively, handle 140 may include a force sensor coupled between the distal end of track 93 and slide block 92.

It also will be evident to one of ordinary skill in the art that any of the handle variations described herein also may incorporate cylindrical extension 141 for insertion into a patient's rectum, one tooth 144 on a ratchet bar to transition the overtube from a flexible state to a rigid state with a single actuation of actuator 27, shield 146 to prevent insertion of the handle into the patient's rectum, indicator 147 to provide a clinician with information about the rigidity of the overtube, and combinations thereof.

Referring now to FIGS. 14A-14C, yet another alternative variation of a tensioning mechanism suitable for use with the apparatus of the present invention is described. Handle 160 is adapted to reconfigure the overtube between its flexible and rigid states with successive actuations of actuator 27. Handle 160 has housing 161 containing plurality of fixed pillars 162 that are circumferentially and azimuthally disposed around inner cylindrical chamber 163 of housing 161. Each fixed pillar 162 has beveled concavity 164 disposed on a proximal end adjacent beveled arm 165. Channel 166 is disposed between adjacent pillars 162.

Handle 160 also incorporates compression spring 167 proximally disposed to bias rotatably mounted manifold 168 against plurality of pillars 162. Manifold 168 incorporates plurality of distally projecting posts 169 having beveled distal ends 170 with inclination angles that match those of beveled concavities 164 and beveled arms 165. Accordingly, when beveled distal ends 170 are forcefully engaged with beveled concavities 164, a component of the force imparted by posts 169 causes manifold 168 to rotate, absent the presence of beveled arm 165. Likewise, when the beveled distal ends are engaged with beveled arms 165, a component of the force imparted by posts 169 rotates the manifold so that pillars 162 are disposed at the proximal ends of channels 166.

Also attached to manifold 168 is tension spring 171, that in turn preferably is coupled to one of the pulley systems of FIGS. 10A-10C or 12A-12D. Tension spring 171 maintains tension wires 90 in constant tension if nestable elements disposed within the overtube slightly shift when the overtube is rigidized. Accordingly, this reduces the risk of reconfiguration of the overtube into the flexible state that otherwise would occur absent tension spring 171.

Handle 160 further comprises translatable cylindrical collar 172 having proximally projecting teeth 173. Each tooth has an inclination angle that substantially is equivalent to that of beveled distal ends 170 of manifold 168. Accordingly, when teeth 173 are engaged forcefully with beveled distal ends 170, a component of the force imparted by the teeth rotates the manifold. Also coupled to collar 172 is actuator 27, illustratively translatable hand grip 174, that may be squeezed against stationary hand grip 175 to retract collar 172 in the proximal direction to contact beveled distal ends 170 of manifold 168.

FIG. 14B depicts the configuration of handle 160 when an overtube coupled thereto is in the rigidized state. Beveled distal ends 170 of manifold 168 are engaged within concavities 164 of pillars 162. When it is desired to reconfigure the overtube into its flexible state, translatable hand grip 174 is squeezed against stationary hand grip 175. This action translates collar 172 in the proximal direction. When teeth 173 engage beveled distal ends 170, continual proximal advancement of translatable hand grip 174 causes collar to push manifold 168 in the proximal direction against compression spring 167. When beveled distal ends 170 clear beveled arm 165, the forces imparted by teeth 173 to the beveled distal ends rotate manifold 168 so that beveled distal ends 170 are engaged to beveled arms 165, as shown in FIG. 14B.

Retraction of collar 172 disengages teeth 173 from manifold 168. The forces imparted by beveled arm 165 to the beveled distal ends rotate manifold 168 until the beveled distal ends clear pillar 162. Thereafter, the bias of compression spring 167 advances plurality of posts 169 into channels 166. FIG. 14C depicts this configuration, in which the overtube is in its flexible state.

To reconfigure the overtube back into its rigid state, translatable hand grip 174 again is squeezed against stationary hand grip 175. This proximally advances collar 172 until teeth 173 contact beveled distal ends 170 of posts 169. Continual proximal actuation of translatable hand grip 174 causes collar 172 to push posts 169 out of channels 166. When beveled distal ends 170 clear pillars 162, the forces imparted by teeth 173 to beveled distal ends 170 rotate manifold 168. Distal retraction of collar 172 disengages teeth 173 from manifold 168, and the bias of compression spring 167 advances manifold 168 until beveled distal ends 170 completely engage concavities 164.

Referring now to FIG. 15, still another alternative variation of handle 21 suitable for use with the apparatus of the present invention is described. Handle 180 comprises housing 181 containing lumen 25 of the overtube. Handle 180 further includes piston 182 translatably disposed within piston housing 183, which is coupled in pneumatic communication via port 184 and tube 185 with a pressure source (not shown). Attached to piston shaft 186 is pulley 187 around which proximal tension wire 188 is disposed. Proximal tension wire 188 is affixed to housing 181 at its proximal end 189 and to tension spring 190 at its distal end. Preferably, tension spring 190 distally is coupled to one of the pulley systems of FIGS. 10A-10C or 12A-12D. Similar to tension spring 171 of FIGS. 14A-14C and compression springs 98 of FIGS. 11 and 13, tension spring 190 maintains the tension wires in constant tension when the overtube is in the shape-locked state. This reduces the risk of reconfiguration of the overtube to its flexible state if nestable elements disposed therein slightly shift relative to adjacent nestable elements.

To stiffen the overtube, the pressure source may be actuated to infuse piston housing 183 with pressurized air that proximally advances piston 182. This in turn advances pulley 187 in the proximal direction, so that tension is applied to proximal tension wire 188. That tension is transmitted through tension spring 190 to tension wires disposed within the overtube, thereby imposing a compressive clamping load to adjacent nestable elements disposed within the overtube. To transition the shape-locked overtube into the flexible state, the pressure source may be actuated to remove air from piston housing 183. This retracts piston 182 and pulley 183 in the distal direction, thereby releasing the compressive clamping load applied to the overtube.

Pursuant to another aspect of the present invention, tension spring 190 may be replaced with a damper per se known in the art. In addition to the advantages provided by the tension spring, the damper allows tension within proximal tension wire 188, and thus tension wires disposed within the overtube, to be slowly released. Applicants contemplate that a damper may replace any of the compression and tension springs described herein.

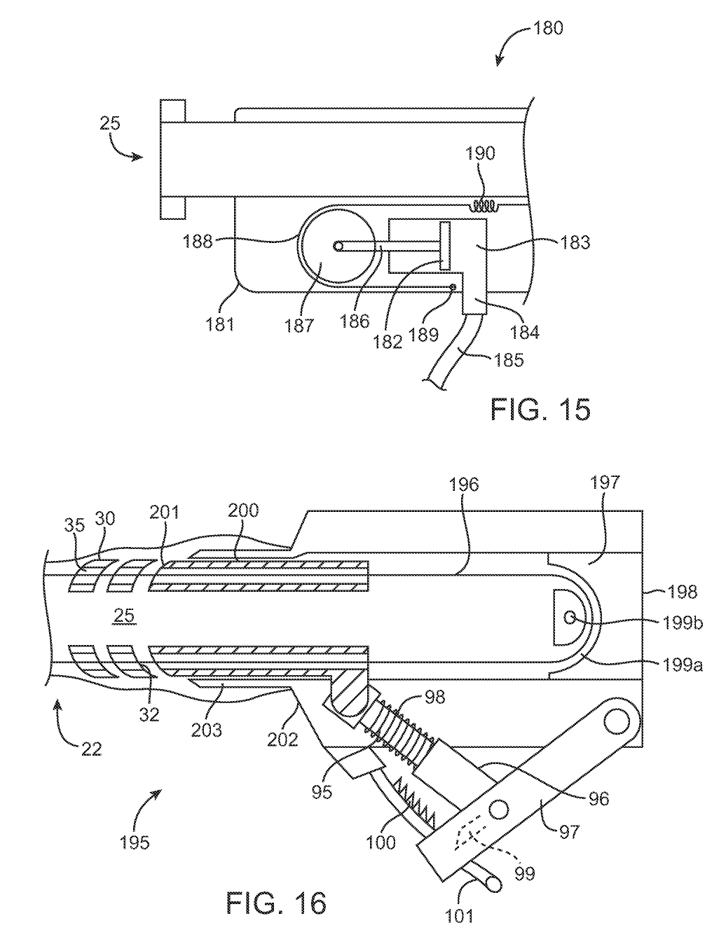

Referring now to FIG. 16, apparatus 20 may be provided with a tensioning mechanism that is selectively operable to transition overtube 22 between the flexible and rigid states substantially without proximal movement of distal region 23 (see FIG. 2). In FIG. 16, tension wire 196 and lumen 25 extend from distal region 23, through overtube 22, and to handle 195. Within handle 195, tension wire 196 is slidably coupled to pulley manifold 197 that is rigidly or rotatably affixed to distal end 198 of the handle. Pulley manifold 197 preferably includes orthogonally disposed first and second channels 199a and 199b. While FIG. 16 depicts only one tension wire, it should be understood that a second tension wire preferably is disposed through second channel 199b and nestable elements 30.

Similar to the tensioning mechanisms of FIGS. 12A-12D and 13, the present tensioning mechanism also provides overtube 22 with a fail-safe mode. If the tension wires disposed through channels 199 comprise two independent wires, the load within overtube 22 remains symmetrically distributed when one of the wires breaks. Thus, the risk of reconfiguration of overtube 22 is reduced. If these tension wires comprise a single length of wire, overtube 22 will relax into the flexible state if the single length of wire breaks.

Between pulley 197 and nestable elements 30, tension wire 196 also extends through collar 200, which has distal surface 201 that is contoured to mate with proximal surface 32 of the proximal-most nestable element 30. Collar 200 is disposed to translate within housing 202 so that distal surface 201 engages proximal surface 32 of nestable element 30 when collar 220 is advanced in the distal direction.

Collar 200 pivotally is connected to plunger 95, which is slidably disposed within plunger housing 96. Plunger housing 96 in turn is mounted pivotally to actuator 27, illustratively hand grip 97. To bias hand grip 97 against actuation absent an externally applied force, and to maintain constant tension within tension wire 196 when overtube 22 is rigidized, compression spring 98 is provided concentrically disposed about plunger 95.

Hand grip 97 also includes pawl 99, which is disposed to engage teeth 100 on ratchet bar 101 to prevent proximally-directed motion of collar 200. Ratchet bar 101 pivotally is mounted in housing 202 with a spring (not shown) that, with the aid of compression spring 98, biases pawl 99 against teeth 100 of ratchet bar 101. Handle 195 also may incorporate annular extension 203 that is disposed surrounding collar 200 and that may be inserted into a patient's rectum.

Similar in operation to handle 91 of FIG. 11, when hand grip 97 is squeezed, pawl 99 engages the next distal-most tooth 100. This action also transmits force through compression spring 98, which pushes collar 200 into engagement with the proximal-most nestable element. Continual actuation of hand grip 97 causes collar 200 to exert an increasing compressive clamping load to nestable elements 30, which causes overtube 22 to stiffen into its shape-locked state.

Advantageously, this configuration permits overtube 22 to reconfigure between the flexible and rigid states without substantial proximal movement of the distal end of the overtube. In previous variations, nestable elements 30 are advanced in the proximal direction when overtube 22 is rigidized, and due to compression of adjacent nestable elements, overtube 22 shortens in length. In contrast, when the present variation advances the nestable elements in the distal direction, overtube 22 maintains its length despite compression of adjacent nestable elements since the length of the overtube substantially is limited by the length of tension wire 196. This provides greater accuracy when using the apparatus of the present invention, and is particularly useful in delicate procedures.

It will be apparent to one of ordinary skill in the art that, similar to the tensioning mechanism described in reference to FIG. 13, ratchet bar 101 may be provided with only one tooth. Alternatively, with minor modifications that will be evident to one of ordinary skill in the art, the tensioning system of FIGS. 14A-14C may be coupled to collar 200 to transition overtube 22 between the flexible and rigid states with successive actuations of actuator 27, or the piston mechanism described in reference to FIG. 15 may be coupled to collar 200 to drive translation thereof. More specifically, rather than being pivotally coupled to plunger 95, collar 200 instead may be fixedly coupled to a piston disposed to provide motion along the longitudinal axis of collar 200. Furthermore, second channel 199b may be eliminated from pulley manifold 197 so that a single tension wire may translatably extend through first channel 199a and diametrically disposed tension wire bores disposed within collar 200 and nestable elements 30. When the single tension wire breaks, the overtube relaxes into the flexible state immediately, thereby providing a fail-safe mode that reduces the risk of undesired reconfiguration of the overtube.

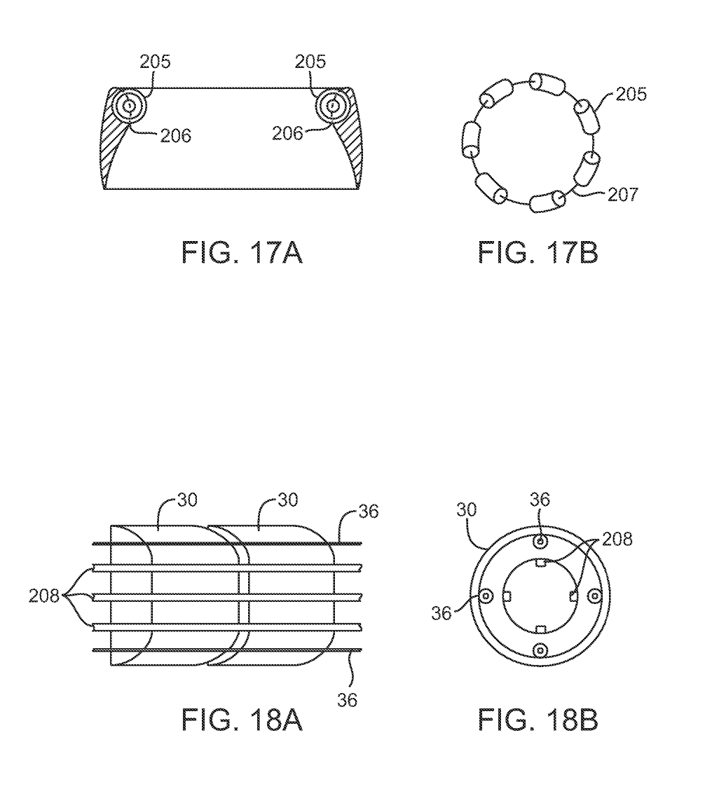

With respect to FIGS. 17A and 17B, an alternative structure is described to facilitate movement of a colonoscope within lumen 25 of overtube 22. In particular, instead of using inner lining 43 as depicted in FIG. 4, some or all of nestable elements 30 may include roller bearings 205 that are received in insets 206 formed in nestable elements 30. Bearings 205 may be disposed on ring 207 to facilitate assembly of the device.

FIGS. 18A and 18B depict a further alternative variation, in which lubricious flexible rails 208 are disposed within bore 33 of nestable elements 30. Rails 208 span the length of lumen 25, and reduce contact between the colonoscope and the interior of the overtube, thereby facilitating movement of the colonoscope through overtube 22.

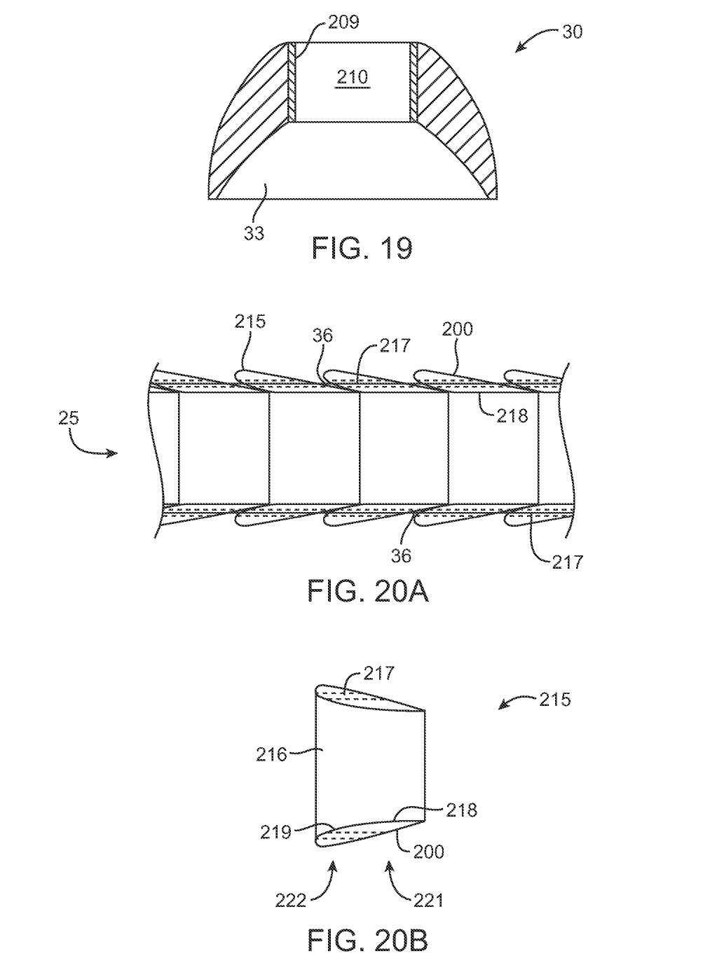

In FIGS. 19 and 20, still further alternative structures are described to facilitate movement of a colonoscope within lumen 25 of overtube 22. More specifically, rather than using liner 43 as shown in FIG. 4, some or all of nestable elements 30 may incorporate hydrophilically-coated polymeric layer 209, which may be disposed surrounding distal portion 210 of bore 33.

Alternatively, as described in FIGS. 20A and 20B, overtube 22 may comprise multiplicity of frustoconical elements 215 that, when nested, provide a smooth inner lumen to accommodate colonoscope 10 without the need for a separate liner. Each frustoconical element 215 includes central bore 216, and at least two or more tension wire bores 217. Central bore 216 is defined by cylindrical distal inner surface 218 that has a substantially constant diameter, and proximal inner surface 219 that is continuous with distal inner surface 218.

Proximal inner surface 219 is slightly curved in a radially outward direction so that, when tension wires 36 are relaxed, proximal inner surface 219 can rotate relative to external surface 220 of an adjacent element. External surface 220 of each frustoconical element may be straight or contoured to conform to the shape of proximal inner surface 219, and tapers each element so that distal end 221 is smaller in outer diameter than proximal end 222. When frustoconical elements 215 are nested together, distal inner surface 218 of each frustoconical element is disposed adjacent to the distal inner surface of an adjoining frustoconical element.

Advantageously, the present configuration provides lumen 25 with a substantially continuous profile. This permits smooth advancement of colonoscope 10 therethrough, and thereby eliminates the need to dispose a separate liner within lumen 25. To provide a lubricious passageway to further facilitate advancement of the colonoscope, each frustoconical element optionally may incorporate an integral hydrophilic polymeric lining as described with respect to the preceding variation of FIG. 19, or a thin, flexible lining having a hydrophilic coating may be disposed through lumen 25.

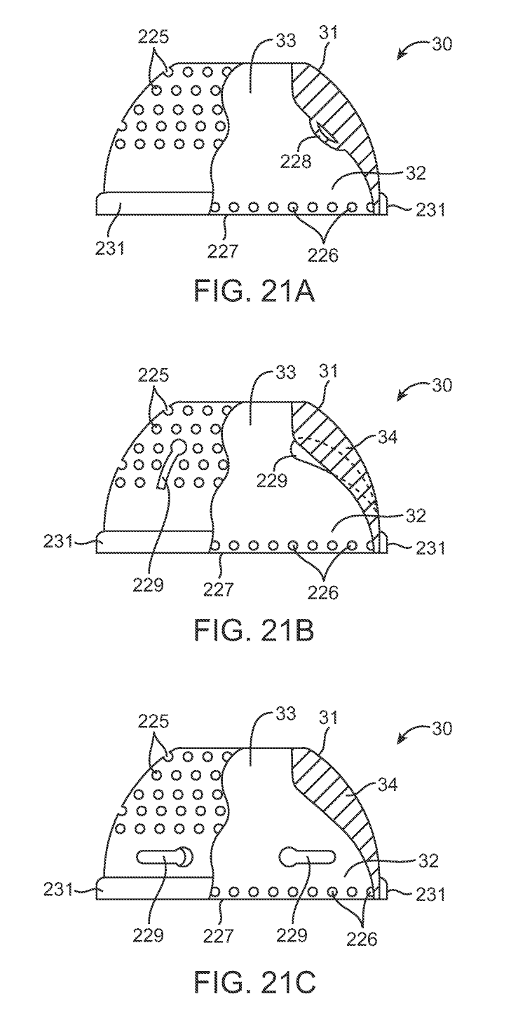

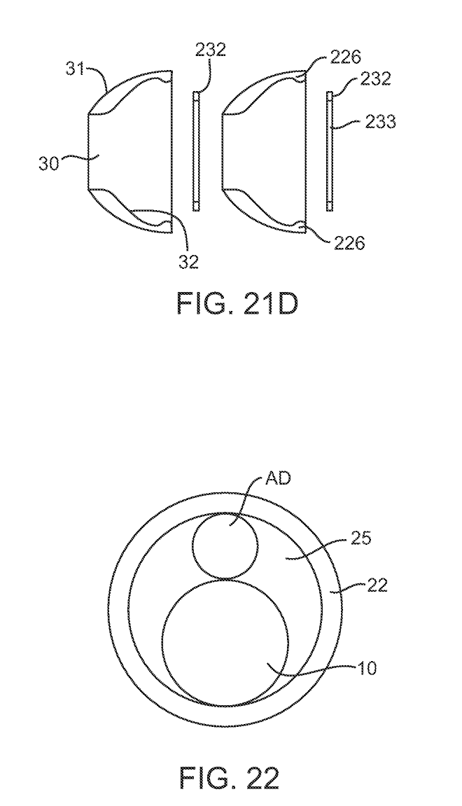

In FIGS. 21A-21C, yet another alternative structure is described, in which distal surface 31 of each nestable element is macroscopically textured to increase the friction between adjacent nestable elements 30 when a compressive clamping load is applied to overtube 22. Illustratively, each element 30 may incorporate multiplicity of divots 225 disposed on distal surface 31, and teeth 226 that are disposed on proximal surface 32 adjacent proximal edge 227. Teeth 226 are contoured to mate with the multiplicity of divots disposed on an adjacent element. Accordingly, when overtube 22 is tensioned, retraction of tension wires 36 (see FIG. 3) applies a clamping load to elements 30 that causes teeth 226 of each element to forcefully engage divots 225 of an adjacent element. This reduces the risk of relative angular movement between adjacent nestable elements 30 when overtube 22 is shape-locked, which in turn reduces the risk of undesired reconfiguration of the overtube.

To prevent divots 225 and teeth 226 from engaging, and thus provide smooth angular movement between adjacent elements 30, when overtube 22 is in the flexible state, one or more leaf springs 228 may be molded integrally with proximal surface 32. Accordingly, absent compressive clamping load applied by tension wires 36 to stiffen overtube 22, leaf spring 228 of each element 30 coacts with distal surface 31 of an adjacent element to prevent coaction of proximal and distal surface 32 and 31, which prevents engagement of teeth 226 with divots 225.

Alternatively, rather than having a leaf spring, nestable elements 30 may be provided with one or more cantilever springs 229 that are cut from wall 34 and plastically bent into bore 33 of nestable element 30. Similar to leaf springs 228, cantilever springs 229 prevent coaction between distal and proximal surfaces 31 and 32 so that teeth 226 do not engage divots 225 absent a compressive clamping load. Cantilever springs 229 may be aligned with a longitudinal axis of nestable element 30, as shown in FIG. 21B, and/or aligned with a circumference of nestable element 30, as shown in FIG. 21C. Applicants also contemplate that teeth 226 may be disposed on distal surface 31 and divots 225 may be disposed on proximal surface 32. One of ordinary skill in the art will recognize additional macroscopic textures that will increase friction between distal and proximal surfaces of adjacent elements 30.

On the other hand, instead of providing leaf or cantilever springs integral with nestable elements 30, thin, flexible disc 232 (FIG. 21D) may be disposed between adjacent nestable elements 30 to prevent divots 225 (see FIGS. 21A-21C) and teeth 226 of the adjacent elements from engaging, absent a compressive clamping load. Each disc 232 incorporates central bore 233 that accommodates a colonoscope, and is made from an elastomeric material. For purposes of illustration, nestable elements 30 and discs 232 are shown spaced-apart, but it should be understood that the elements and discs are disposed so that distal surface 31 of one element 30 and proximal surface 32 of an adjacent element coacts with disc 232, which is disposed therebetween. It also should be understood that each nestable element 30 also comprises tension wire bores, which are not shown in FIGS. 21A-21D for illustrative purposes.

Pursuant to one aspect of the present invention, nestable elements 30 also may incorporate band 231 that is disposed distally adjacent to proximal edge 227. Band 231 increases the thickness of the proximal portion of wall 34 to distribute the applied compressive clamping load over a larger cross-sectional area, and thereby reduce radially outward deflection of wall 34. This in turn reduces longitudinal contraction of overtube 22. Band 231 preferably is made from a metal to provide greater structural integrity to wall 34, but also may be integral therewith.

In accordance with another aspect of the present invention, the diameter of lumen 25 preferably is configured to facilitate simultaneous passage of more than one diagnostic or therapeutic instrument therethrough. As shown in FIG. 22, lumen 25 may be dimensioned to permit auxiliary devices AD, such as for aspiration, biopsy, or additional lighting, to be advanced alongside colonoscope 10. For example, if lumen 25 has a diameter of 13 mm and colonoscope 10 has an outer diameter of 10 mm, auxiliary device AD, such as a catheter, having a diameter of between 3F to 9F may be advanced through the remaining space within lumen 25. Advantageously, this permits auxiliary devices AD to be successively placed within the patient's colon to perform additional diagnostic or therapeutic procedures without the need to remove colonoscope 10 and overtube 22 therefrom.

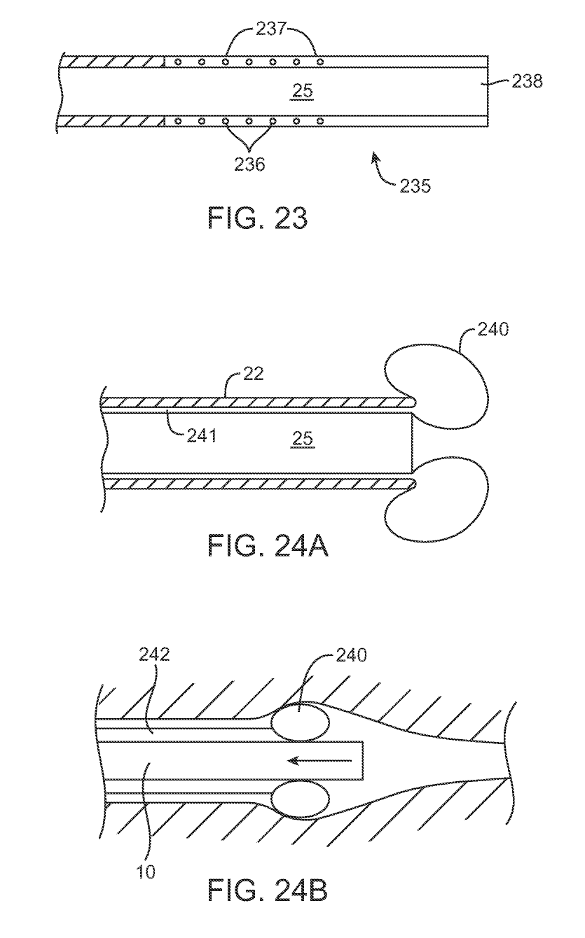

Referring to FIG. 23, an alternative variation of a distal region suitable for use in the overtube of the present invention is described. Distal region 235 is similar in construction to distal region 23 of the variation of FIG. 4, but has flexible coil 236 embedded in only the proximal portion of elastomeric layer 237. Atraumatic tip 238 at the distal end of distal region 235 may further enhance the steerability of overtube 22 when the steerable tip of the colonoscope is disposed therein.

FIGS. 24-28 illustrate additional configurations of atraumatic tips suitable for causing “tenting” of the wall of the hollow body organ. As used herein, tenting refers to the tendency of the atraumatic tip to be deflected radially outward in the vicinity of the tip of the overtube. This reduces the risk that the wall of the organ will become pinched or caught between the colonoscope and the entry to overtube 22 when the colonoscope is retracted within the overtube.

FIG. 24A shows atraumatic tip 24 in the form of an inflatable donut-shaped balloon 240 affixed to distal region 23 of overtube 22. Inflation lumen 241 extends from the handle through overtube 22 to provide fluid communication between balloon 240 and an inflation source, such as a syringe (not shown). As illustrated in FIG. 24B, when balloon 240 is inflated, the wall of the colon radially deflects around balloon 240. Thus, when colonoscope 10 is retracted into lumen 25, it is less likely that the wall of the colon will be pinched or potentially dissected between overtube 22 and colonoscope 10. Furthermore, when inflated, balloon 240 closes annular gap 242 disposed between the wall of overtube 22 and colonoscope 10 to prevent bodily fluids and other matter from entering lumen 25. Advantageously, balloon 240 provides a custom fit around colonoscope 10.

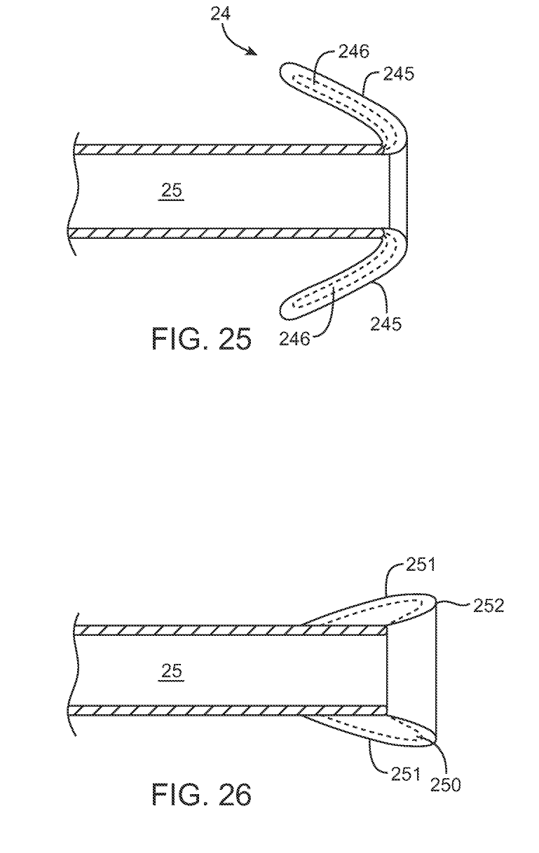

FIG. 25 depicts a further alternative variation of atraumatic tip 24, comprising soft membrane 245 covering shape memory alloy petals 246. Petals 246 preferably comprise loops of shape memory alloy wire, e.g., nickel titanium alloy, and extend radially outward in the proximal direction near the distal opening into lumen 25, so that the proximal end of membrane-covered petals causes the “tenting” effect described hereinabove. The shape memory alloy may be activated to adopt a pre-formed shape when exposed to body temperature, and returned to a contracted state by flushing overtube 22 with cold water or air. Alternatively, petals 246 may be mechanically extended or retracted, or self-expanding.

FIG. 26 depicts a further alternative variation of atraumatic tip 24. In the variation of FIG. 26, petals 250 covered by soft elastomeric membrane 251 extend distally from distal region 23 to form funnel-shaped element 252. Atraumatic tip 24 provides a similar tenting effect to that described for the preceding variations.

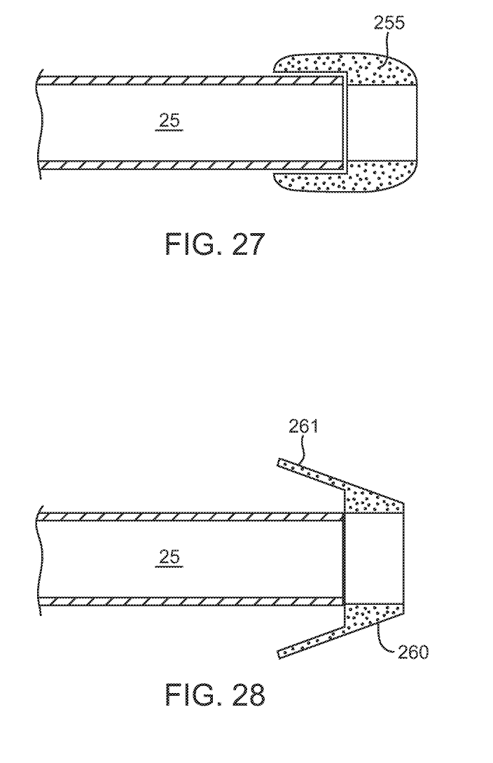

FIGS. 27-28 provide further alternative configurations for atraumatic tip 86 of the variation of FIG. 9. Tip 255 preferably comprises a foam or soft elastomer, and may be affixed to distal region 23 of overtube 22 using a suitable biocompatible adhesive. FIG. 28 depicts an alternative shape for a foam or soft elastomer bumper 260, which includes a proximally-extending flange 261. Of course, one of ordinary skill in the art will recognize that other configurations may be used in accordance with the principles of the present invention to form atraumatic tips that cause localized tenting of the colon wall, and these atraumatic tips may be used with the passively-steerable distal regions of the variations of FIGS. 4 and 23.

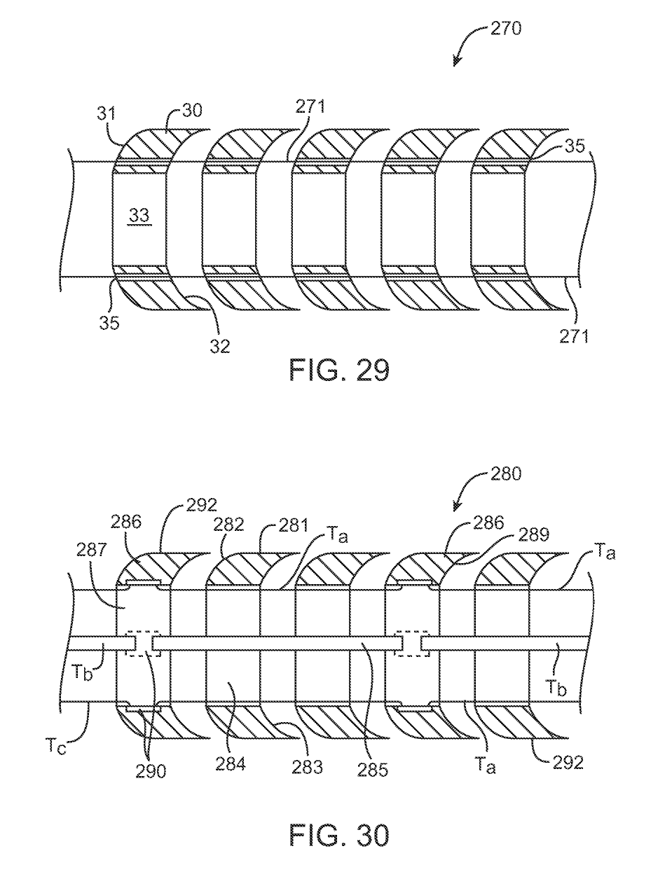

Referring now to FIGS. 29 and 30, alternative variations of the overtube are described. Unlike overtube 22 of previously described variations, in which a mechanical mechanism is actuated to impart a clamping load to a multiplicity of nestable elements, the variations of FIGS. 29 and 30 use alternative tensioning mechanisms. In particular, the following variations comprise a multiplicity of links to which a compressive clamping load may be applied by contraction of shape memory materials.

In FIG. 29, a first alternative variation of the overtube of the present invention is described. Overtube 270 includes multiplicity of nestable elements 30 identical to those described hereinabove. For purposes of illustration, nestable elements 30 are shown spaced-apart, but it should be understood that elements 30 are disposed so that distal surface 31 of each element 30 coacts with proximal surface 32 of an adjacent element. Each of nestable elements 30 has central bore 33 to accommodate colonoscope 10, and preferably two or more tension wire bores 35. When assembled as shown in FIG. 29, nestable elements 30 are fastened with distal and proximal surfaces 31 and 32 disposed in a coacting fashion by a plurality of tension wires 271 that extend through tension wire bores 35.

In contrast to overtube 22 of the previous variations, tension wires 271 of the present overtube are made from a shape memory material, e.g., nickel titanium alloy or an electroactive polymer known in the art. Tension wires 271 are fixedly connected to the distal end of overtube 270 at the distal ends and fixedly connected to handle 21 at the proximal ends. When an electric current is passed through tension wires 271, the wires contract in length, imposing a compressive clamping load that clamps distal and proximal surfaces 31 and 32 of nestable elements 30 together at the current relative orientation, thereby fixing the shape of overtube 270. When application of electrical energy ceases, tension wires 271 re-elongates in length to provide for relative angular movement between nestable elements 30. This in turn renders overtube 270 sufficiently flexible to negotiate a tortuous path through the colon.

To provide overtube 270 with a fail-safe mode that reduces the risk of undesired reconfiguration of the overtube in the event of tensioning mechanism failure, diametrically disposed tension wires 271 may be coupled in a serial circuit. Accordingly, when one wire fails, the wire disposed diametrically opposite also re-elongates to maintain a symmetrical clamping load within overtube 270. Alternatively, all tension wires 271 may be electrically coupled in a serial electrical circuit. Accordingly, when one of the tension wires fails, overtube 270 returns to the flexible state.

It should be understood that a tension spring (not shown) or damper (not shown) that are similar to those described hereinabove may be coupled between the proximal ends of tension wires 271 and handle 21 (see FIG. 2). Inter alia, this maintains the tension wires in constant tension when the overtube is in the shape-locked state, thereby reducing the risk of reconfiguration of the overtube to its flexible state if nestable elements disposed therein slightly shift relative to adjacent nestable elements.

Alternatively, as described in FIG. 30, overtube 280 may include multiplicity of nestable elements 281 that are similar to those of the preceding variations. For purposes of illustration, nestable elements 281 are shown spaced-apart, but it should be understood that elements 281 are disposed so that distal surface 282 of each element 280 coacts with proximal surface 283 of an adjacent element. Each of nestable elements 280 has central bore 284 to accommodate colonoscope 10.

When assembled as shown in FIG. 30, nestable elements 280 are fastened with distal and proximal surfaces 282 and 283 disposed in coacting fashion by plurality of thin tension ribbons 285 that are fixedly connected to nestable bridge elements 286. Tension ribbons 285 are made from a shape memory material, e.g., nickel titanium alloy or an electroactive polymer, and may be transitioned from an equilibrium length to a contracted length when electrical current is passed therethrough.

Nestable bridge elements 286 are disposed within overtube 280 between a predetermined number of nestable elements 281. Similar to nestable elements 281, bridge elements 286 also comprise central bore 287 that accommodates colonoscope 10, distal surface 288 that coacts with proximal surface 283 of a distally adjacent nestable element, and proximal surface 289 that coacts with distal surface 282 of a proximally adjacent nestable element 281. Each bridge element also incorporates plurality of conductive elements 290 that are disposed azimuthally around central bore 287, and that preferably couple tension ribbons 285 occupying the same angular circumferential position within overtube 280 in a serial electrical circuit.

When an electrical current is passed through tension ribbons 285, the ribbons contract in length, imposing a compressive load that clamps distal and proximal surfaces of adjacent nestable elements together at the current relative orientation, thereby fixing the shape of overtube 280. When the energy source ceases providing electricity, tension ribbons 285 re-elongates to the equilibrium length to provide for relative angular movement between the nestable elements. This in turn renders overtube 280 sufficiently flexible to negotiate a tortuous path through the colon.

Pursuant to another aspect of the present invention, tension ribbons 285 that are disposed at diametrically opposite circumferential positions may be electrically coupled in a serial circuit. Advantageously, this configuration provides overtube 280 with a fail-safe mode that reduces the risk of undesired reconfiguration of the overtube in the event that one of the electrical circuits established through the tension ribbons is de-energized.

For example, overtube 280 of FIG. 30 may be provided with four sets of tension ribbons equidistantly disposed at 90o intervals. In the event that tension ribbons Ta de-energize, absent electrical communication between tension ribbons Ta and tension ribbons Tc disposed diametrically opposite thereto, overtube 280 will spontaneously reconfigure into a new rigidized shape since the tension within the overtube no longer will be symmetrically balanced. The new shape of overtube 280 may not replicate the tortuous path of the colon, and thus may cause substantial harm to the patient.

Advantageously, the present invention may reduce the risk of undesired reconfiguration preferably by electrically coupling diametrically disposed tension ribbons in a serial circuit. When tension ribbons Ta are de-energized, tension ribbons Tc also de-energize to provide overtube 280 with symmetrical tension, as provided by tension wires Tb and the tension wires disposed diametrically opposite thereto (not shown). In this manner, the overtube retains its desired rigidized shape in the event that the tensioning mechanism malfunctions. To immediately return overtube 280 to its flexible state in the event that any of the tension ribbons are de-energized, all tension ribbons 285 may be electrically coupled in a serial circuit.

In an alternative variation, tension ribbons 285 may be electrically coupled to rigidize select regions of the overtube without rigidizing the remainder of the overtube. Illustratively, this may be accomplished by coupling longitudinally adjacent tension ribbons in a parallel circuit, and circumferentially adjacent tension ribbons in a serial circuit.

Of course, it will be evident to one of ordinary skill in the art that, while FIG. 30 depicts tension ribbons 285 to be disposed within central bores 284 and 287, the tension ribbons also may be disposed adjacent external lateral surfaces 292 of nestable elements 281 and 286. Alternatively, the tension ribbons may extend through tension ribbon bores (not shown) that may extend through the distal and proximal surfaces of nestable elements 281, and be affixed to nestable bridge elements 286.

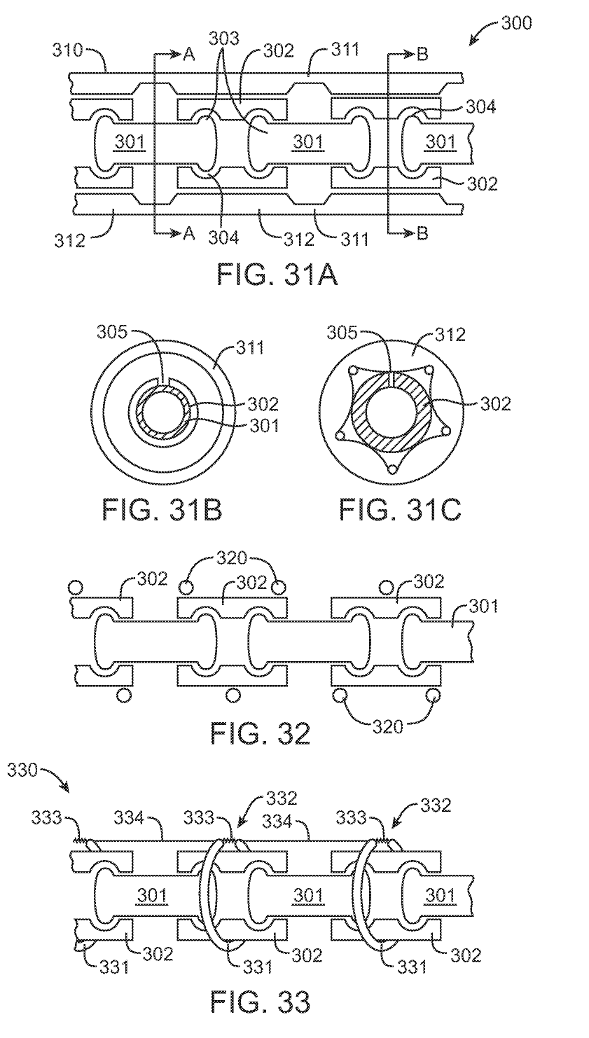

With respect to FIGS. 31-37, alternative variations of overtube 22 are described. Unlike overtube 22 of the above-described variations, which comprised a multiplicity of nestable elements that are clamped with a plurality of tension wires or ribbons, the variations of FIGS. 31-37 use alternative clamping mechanisms. In particular, the following variations comprise a plurality of links that may be stiffened by the use of compressive sleeves that compress individual links disposed along the length of the overtube.

Referring now to FIGS. 31A-31C, a fourth alternative variation of the overtube of the present invention is described. Overtube 300 comprises a multiplicity of alternating spool links 301 and clamp links 302. Each spool link 301 and clamp link 302 has a bore disposed therethrough to accommodate a standard colonoscope. Spool link 301 comprises rounded edges 303 disposed on its distal and proximal ends that are contoured to permit limited rotatable engagement with one of two contoured grooves 304 disposed within the bore of clamp link 302. Accordingly, clamp link 302 comprises a greater outer diameter than spool link 301. Each clamp link 302 also has through-wall split 305 longitudinally disposed to permit a reduction in the diameter of clamp link 302 when the clamp link is compressed, as discussed hereinafter.

Still referring to FIGS. 31A-31C, a first variation of a compressive sleeve comprising inflatable sleeve 310 having first compressive portions 311 and second compressive portions 312. Sleeve 310 is configured so that the inner diameters of second compressive portions 312 are smaller than those of first compressive portions 311 when sleeve 310 is inflated. Second compressive portions 312 may be disposed to engage clamp links 302. Thus, when inflatable sleeve 310 is inflated by an inflation source (not shown) coupled to the handle, second compressive portions 312 compress against clamp links 202 to shape-fix overtube 300. In FIGS. 31B and 31C, cross sectional views of first compressive portions 311 and second compressive portions 312, respectively, are shown when sleeve 310 is in its inflated state.

FIG. 32 illustrates an alternative variation of a compressive sleeve that also comprises an inflatable bladder. Unlike inflatable bladder 310 of FIGS. 31A-31C, spiral bladder 320 has a constant inner diameter. Spiral bladder 320 preferably is helically disposed around the overtube. Accordingly, when bladder 320 is inflated, clamp links 302 are compressed onto spool links 301 to stiffen the overtube.

FIG. 33 depicts a further variation of a compressive sleeve 330, comprising discontinuous hoops 331 made of shape memory alloy (e.g. nickel titanium alloy). Each hoop 331 includes gap 332, which is spanned by spring 333. Each hoop 331 is electrically connected to neighboring hoops 331 via insulated wires 334, so that a serial electrical circuit is established. When hoops 331 are energized, they undergo a phase transition that causes the hoops to contract into a preformed shape that is diametrically smaller than the non-energized shape. Since hoops 331 may be disposed about clamp links 302, contraction of hoops 331 may be used to apply a clamping load that compresses links 302 onto spool links 301 to stiffen the overtube.

Springs 333 contribute to structural integrity when hoops 331 are in their non-energized state. To energize and thereby contract hoops 331, an electrical current may be run through wires 334. To return hoops 331 to their non-contracted state and thereby return the overtube to its flexible state, hoops 331 may be flushed with cold water or air. Of course one of ordinary skill in the art will recognize that hoops 331 also may be individually energized, thus requiring a parallel circuit.

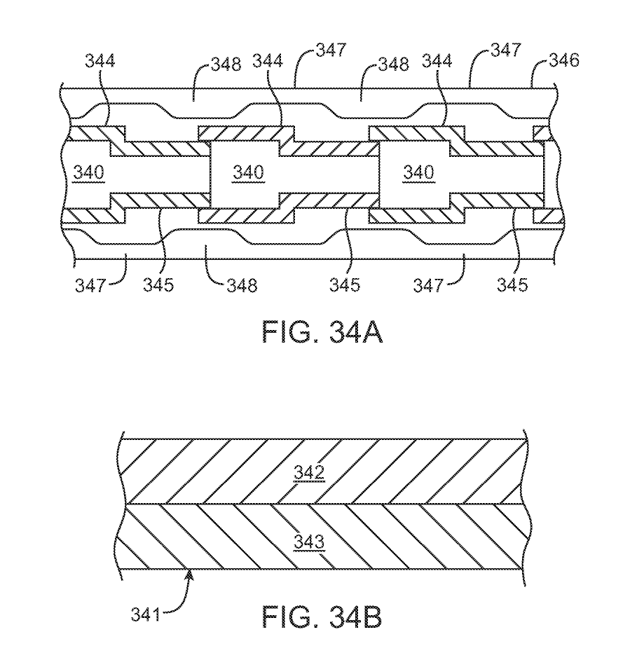

With respect to FIGS. 34A-34B, a still further alternative variation of an overtube suitable for use in the present invention is described. This variation comprises helical links 340 that are formed from an integral strip 341 having regions of different durometer, e.g., rigid material 342 and soft material 343. When strip 341 is helically wound, helical links 340 are formed having rigid portions 344 and soft portions 345. Rigid portions 344 provide structural integrity to the overtube, while soft portions 345 provide flexibility.

Helical links 340 are disposed within compressive sleeve 346, which includes first compressive portions 347 and second compressive portions 348. Compressive sleeve 346 is identical in structure and operation to that described in FIGS. 31A-31C, except that second compressive portions 348 are aligned with, and apply a clamping force to, rigid portions 344 of helical links 340. It will of course be understood that an overtube in accordance with the principles of the present invention could alternatively be formed using helical links 340 and either of the clamping systems described with respect to FIGS. 32 and 33.

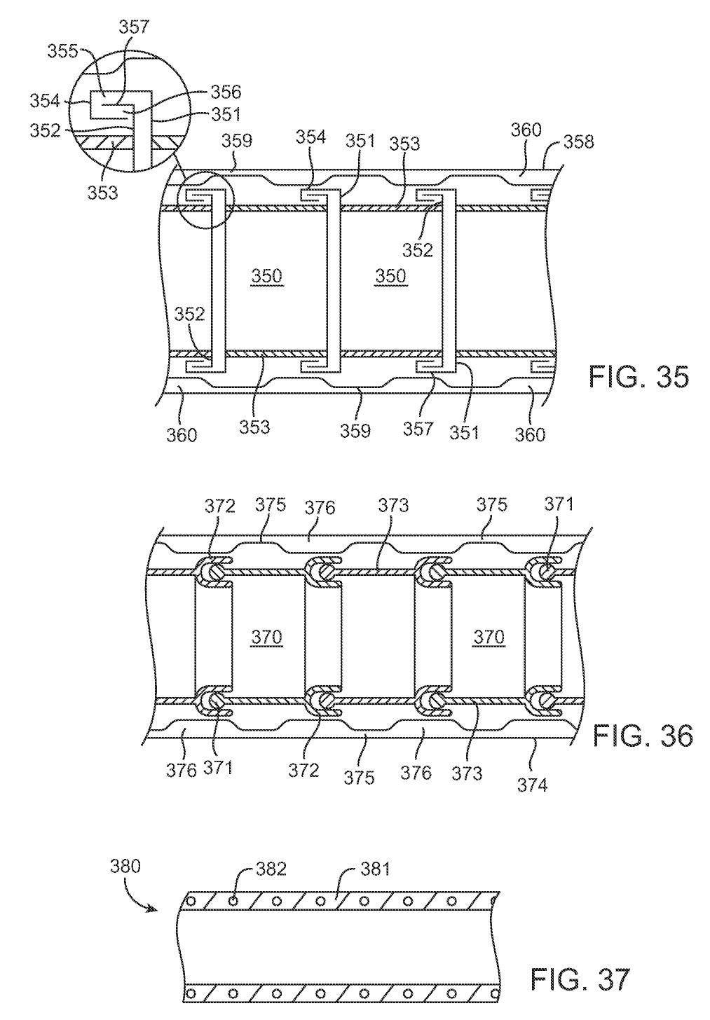

Referring now to FIG. 35, another alternative variation of an overtube is described, in which each Grecian link 350 includes rigid first and second rims 351 and 352 disposed at longitudinally opposing ends of flexible body 353. First rim 351 comprises U-shaped arm 354 that defines channel 355 and opening 356. Second rim 352 includes retroflexed arm 357, which when engaged to first rim 351 of an adjacent, is disposed within channel 355 of U-shaped arm 354 through opening 356 so that U-shaped arm 354 and retroflexed arm 357 are engaged and overlap along the longitudinal axis of the overtube.

Grecian links 350 are disposed within compressive sleeve 358, which includes first compressive portions 359 and second compressive portions 360. Compressive sleeve 358 is identical in structure and operation to that described in FIGS. 31A and 34A, except that second compressive portions 360 are aligned with, and apply a clamping force to, overlapping U-shaped arm 354 and retroflexed arm 357 of the first and second rims. It will of course be understood that an overtube in accordance with the principles of the present invention couple alternatively be formed using Grecian links 350 and either of the clamping systems described with respect to FIGS. 32 and 33.

Referring now to FIG. 36, yet another alternative variation of an overtube suitable for use in the present invention is described. This variation comprises joint links 370 that include ball 371 and socket 372 disposed at longitudinally opposing ends of flexible body 373. When adjacent joint links 370 are engaged, ball 371 of one link is disposed within socket 372 of an adjacent link. When the overtube is flexed, ball 371 coacts with socket 372 to provide articulation of the overtube.

Joint links 370 are disposed within compressive sleeve 374, which includes first compressive portions 375 and second compressive portions 376. Compressive sleeve 374 is identical in structure and operation to that described in FIGS. 31A, 34A and 35, except that second compressive portions 376 are aligned with, and apply a clamping force to, socket 372 within which ball 371 of an adjacent link is disposed. It will of course be understood that an overtube in accordance with the principles of the present invention couple alternatively be formed using joint links 370 and either of the clamping systems described with respect to FIGS. 32 and 33.

With respect to FIG. 37, a still further variation of an overtube suitable for use in the apparatus of the present invention is described. Overtube 380 comprises a heat-softenable polymer layer 381, (e.g., Carbothane®, a proprietary urethane-based polymer available from Thermedics Polymer Products, Woburn, Mass.), having wire 382 embedded within it. Wire 382 is coupled at the handle to an energy source, so that by passing an electric current through wire 382, sufficient resistive heating occurs to soften the polymer layer 381, rendering it sufficiently flexible to negotiate tortuous or unsupported anatomy. When electrical energy is not supplied to wire 382, no resistive heating of the wire or the polymer layer occurs, and the overtube instead cools and stiffens. Wire 382 serves the dual purpose of providing kink resistance and electric heating.

Still referring to FIG. 37, yet another alternative variation of an overtube suitable for use in the present invention comprises a soft elastomeric polymer layer 381 having a shape memory alloy wire 382 embedded within layer 381. In this variation, the shape memory alloy is selected to have a martensite transition temperature above body temperature. When wire 382 is heated to a temperature above body temperature, such as by passing an electric current through it, the wire transitions into the austenitic phase, and becomes stiffer, thereby shape locking the overtube. When application of the electric current ceases, wire 382 cools back into the martensitic phase, and renders the overtube flexible.

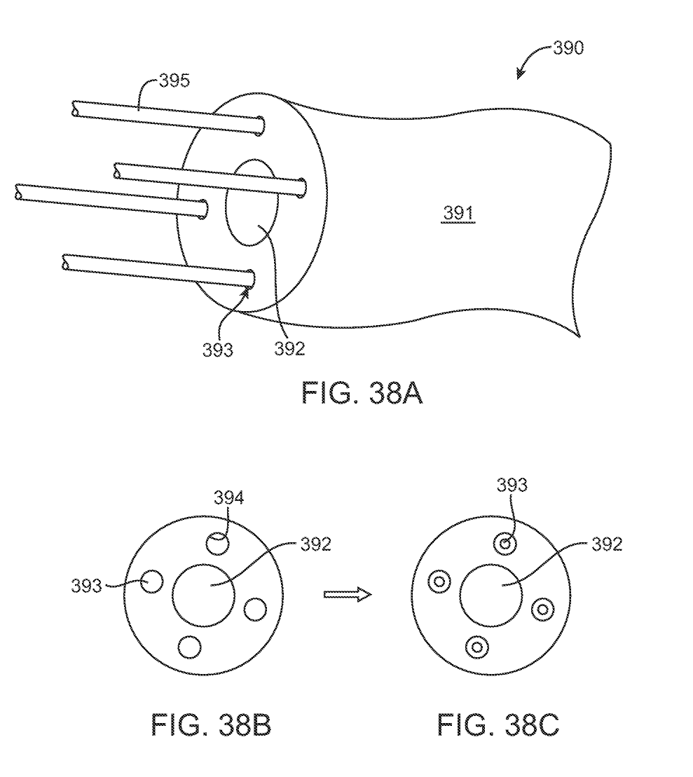

Referring now to FIGS. 38A-38C, an additional alternative variation of an overtube suitable for use with the present invention is described. Overtube 390 comprises elongate body 391 having central lumen 392 that accommodates colonoscope 10, and wire lumens 393 that are defined by cylindrical wire lumen surfaces 394. Within each wire lumen 393 is disposed wire 395 that extends the length of the elongate body. Elongate body 391 is made from an electroactive polymer known in the art that permits wire lumens 393 to vary in diameter responsive to electrical energization.

In particular, when an electrical current is passed through elongate body 391, the diameter of each wire lumen 393 decreases so that the wire lumens clamp around respective wires 395. Preferably, both wires 395 and wire lumen surfaces 394 are textured to enhance friction therebetween. This prevents further relative movement between elongate body 391 and wires 395, and stiffens overtube 390. When application of the electrical current ceases, wire lumens 393 increase in diameter to release wires 395 so that elongate body 391 may shift relative to wires 395. This in turn renders overtube 390 sufficiently flexible to negotiate a tortuous path through the colon.

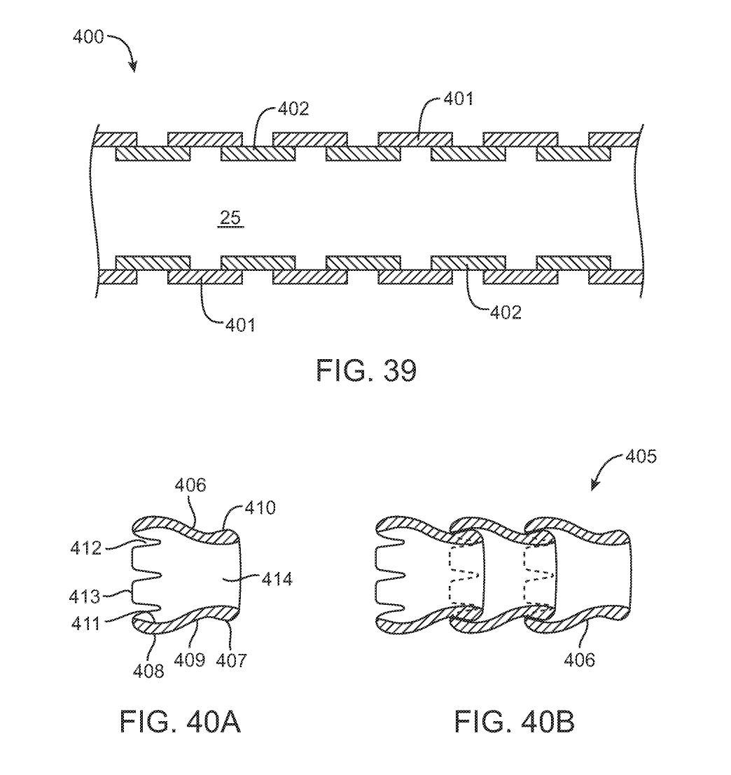

With respect to FIG. 39, yet another alternative variation of the overtube is described. Overtube 400 incorporates multiplicity of variable diameter links 401 disposed in overlapping fashion surrounding multiplicity of rigid links 402, that provide structural integrity to the overtube. Each link comprises a central bore that defines lumen 25 of the overtube, and accommodates a standard commercially available colonoscope. Variable diameter links 401 preferably are manufactured from an electroactive polymer or a shape memory alloy that contract in diameter when energized. When variable diameter links 401 are electrically activated, the variable diameter links tighten about rigid links 402 to transition overtube 400 into a shape-locked state. When the variable diameter links are electrically deactivated, the variable diameter links sufficiently soften to return overtube 400 back to the flexible state.

In a preferred variation, variable diameter links 401 and rigid links 402 are formed from respective strips of material that are helically wound in an overlapping fashion to form overtube 400. Alternatively, each link may be individually formed and disposed in an overlapping fashion.

In FIGS. 40A-40B, still another alternative variation of an overtube suitable for use with the apparatus of the present invention is illustrated schematically. Overtube 405 comprises multiplicity of nestable hourglass elements 406 that preferably are manufactured from an electroactive polymer or a shape memory alloy, and each have bulbous distal and proximal portions 407 and 408 connected by neck 409. The diameter of neck 409 is smaller than the maximum diameter of distal portion 407, which in turn is less than the maximum diameter of proximal portion 408. The distal portion of external surface 410 of each hourglass element 406 is contoured to coact with the proximal portion of internal surface 411 of a distally adjacent hourglass element. Accordingly, when a multiplicity of hourglass elements are nested together to form overtube 405, adjacent elements 406 may move relative to each other when the overtube is in the flexible state.

To reduce friction between adjacent elements during relative movement therebetween, proximal portions 408 include plurality of slits 412 disposed contiguous with proximal edge 413. Slits 412 also facilitate contraction of proximal portion 408 of each element around distal portion 407 of an adjacent element. Each hourglass element 406 also has central bore 414 that accommodates colonoscope 10 (see FIG. 1).

When an electrical current is applied to multiplicity of nestable hourglass elements 406, proximal portion 408 of each element contracts in diameter around distal portion 407 of an adjacent element. The compressive clamping force thereapplied prevents relative movement between adjacent elements, thereby shape-locking the overtube. When the nestable elements are de-energized, proximal portions 408 sufficiently relax to permit relative movement between adjacent nestable elements 406, and thus permit overtube 405 to negotiate tortuous curves. For purposes of illustration, it should be understood that the figures of the present application may not depict an electrolytic medium, electrodes, and insulated wires that are coupled to and facilitate ionization, and thus contraction, of the electroactive polymers described herein.

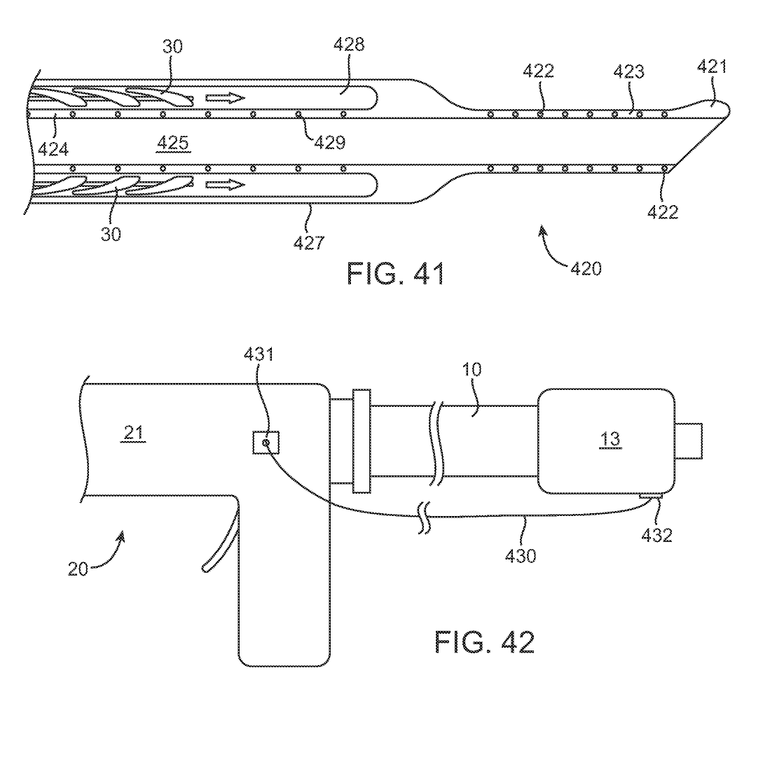

In accordance with another aspect of the present invention, the overtube of the present invention may be provided with disposable sheath 420 that may extend the length of overtube 22 and be removed therefrom. Like the sheath described hereinabove with respect to FIG. 4, sheath 420 of FIG. 41 also incorporates distally-disposed atraumatic tip 421 and flexible, kink-resistant coil 422 encapsulated in flexible layer 423. At its proximal end, layer 423 joins or is integrally formed with lubricious liner 424 defining lumen 425, and flexible elastomeric skin 427. Liner 424 may incorporate optional flexible, kink-resistant coil 429, be made of a thin, flexible material, and/or have a hydrophilic coating thereon, similar to that described in reference to FIG. 4. Between liner 424 and skin 427 is disposed annular chamber 428 within which nestable elements 30 may be inserted. Sheath 420 is configured to slide onto and be removed from a column of nestable elements 30 so that the sheath may be discarded after a single use, while the nestable elements and handle may be sterilized and reused. Advantageously, substantial cost reductions may be realized.

Pursuant to another aspect of the present invention, apparatus 20 further may be provided with a device to secure colonoscope 10 to apparatus 20 prior to insertion of apparatus 20 and colonoscope 10 into the patient. FIG. 42 depicts strap 430 that may be secured distally to handle 21 of apparatus 20 and proximally to proximal portion 13 of colonoscope 10. Strap 430 preferably has a length that prevents colonoscope 10 from decoupling from apparatus 20 after the colonoscope is placed within the overtube. Illustratively, strap 430 may be made of ductile wire or Velcro. If strap 430 is made of ductile wire, the strap may be secured to anchors 431 and 432 respectively disposed on handle 21 and colonoscope 10. Anchor 432 may be integral with, or comprise an adhesive suitable for application to colonoscope 10.

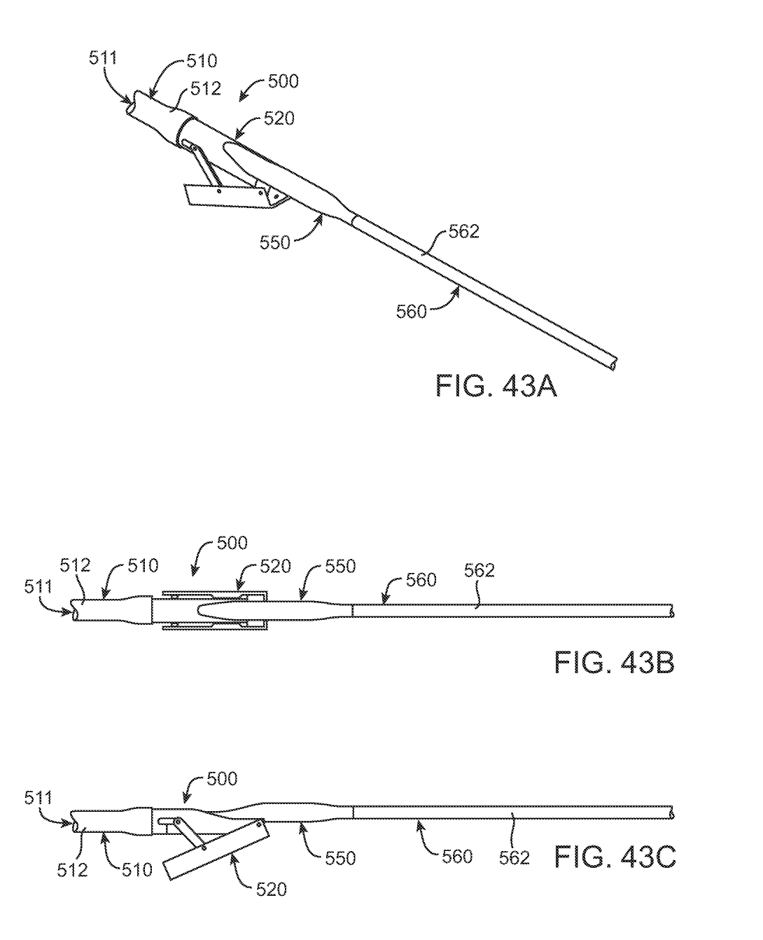

Referring now to FIG. 43, a loading device for inserting a liner within apparatus of the present invention is described. In FIG. 43, apparatus 500 comprises rigidizable/shape-lockable overtube 510, which is coupled to handle 520. Sheath 512 is disposed about the exterior of overtube 510, and lumen 511 extends through the overtube and handle 520. Overtube 510 and handle 520 may, for example, be similar to any handle or overtube described previously hereinabove.

FIG. 43 illustrate apparatus and methods for disposing liner 550, which may comprise a hydrophilic liner and/or may be similar to previously described liner 43 or previously described liner 424, within lumen 511 of apparatus 500. Liner 550 is coupled to a distal end of loading device 560, which illustratively comprises elongate tube or rod 562. Rod 562 preferably is more rigid than liner 550 in order to facilitate placement of the liner.

Liner 550 may be inserted within lumen 511 of apparatus 500 by pushing a proximal end of rod 562 through a distal end (not shown) of overtube 510 and lumen 511. Continued advancement of rod 562 through lumen 511 causes the rod to exit the lumen through proximal handle 520. The rod then may be pulled more proximally until liner 550 exits lumen 511, as seen in FIG. 43. Once inserted within the lumen, liner 550 may be decoupled from loading device 560, for example, by cutting the liner at a position between the proximal end of handle 520 and the distal end of rod 562.

Sheath 512 optionally may be distally coupled to, or integrally formed with, liner 550, and optionally may be advanced over the exterior of overtube 510 concurrent with the advancement of liner 550 through lumen 511. In such a configuration, loading device 560 is utilized to properly position both external sheath 512 and internal liner 550 about apparatus 500. The sheath and liner may, for example, be disposable and may facilitate re-use of overtube 510 and handle 520.

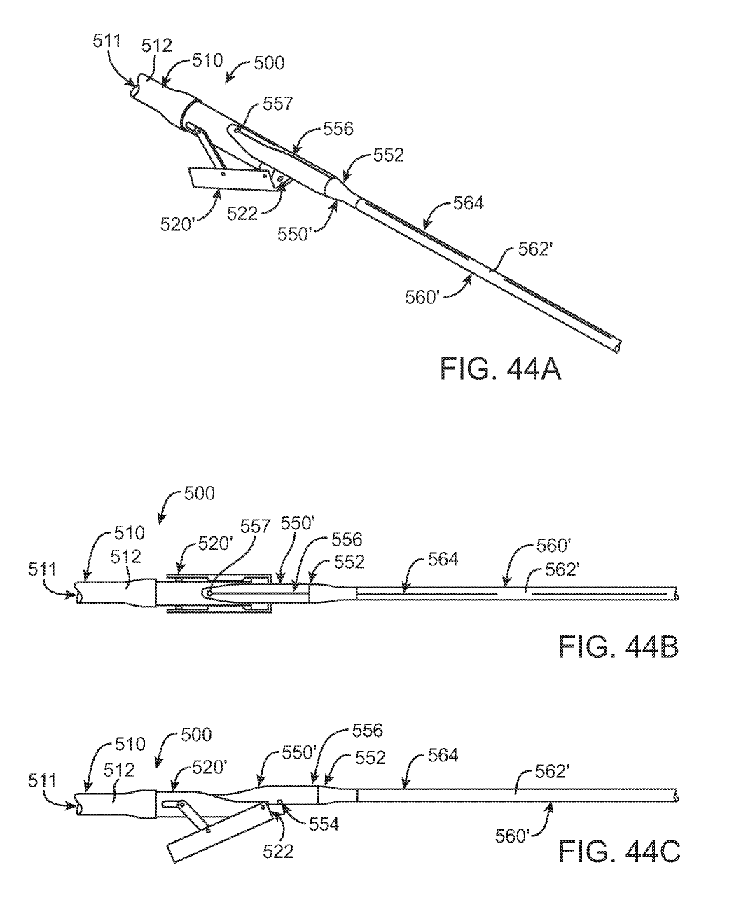

Referring now to FIG. 44, an alternative loading device, liner and apparatus handle are described. Alternative handle 520′ of apparatus 500 comprises anchor post 522. Alternative loading device 560′ comprises rod or tube 562′ having alignment indicator 564. Alternative liner 550′ comprises detachment perforation 552, anchoring hole 554 and access slit 556.

Slit 556 distally terminates at strain relief 557, which reduces a risk of slit 556 propagating distally along the length of liner 550′. Alignment indicator 564 of rod 562′ preferably is aligned with slit 556 to facilitate proper orientation of liner 550′ within apparatus 500, and more specifically within handle 520. As discussed hereinafter, anchoring hole 554 of liner 550′ preferably is aligned with anchoring post 522 of handle 520′ for securing the liner to the handle.

Liner 550′ may be disposed within lumen 511 of apparatus 500 in a manner similar to that described previously with respect to liner 550 of FIG. 43. The liner may be inserted within the lumen by pushing a proximal end of rod 562′ through a distal end of overtube 510 and lumen 511 until the rod exits the lumen through handle 520′. The rod then may be pulled more proximally until liner 550′ exits the lumen, as in FIG. 44. Preferably, alignment indicator 564 is utilized while proximally pulling rod 562′ in order to properly align liner 550′ with apparatus 500 and handle 520′.

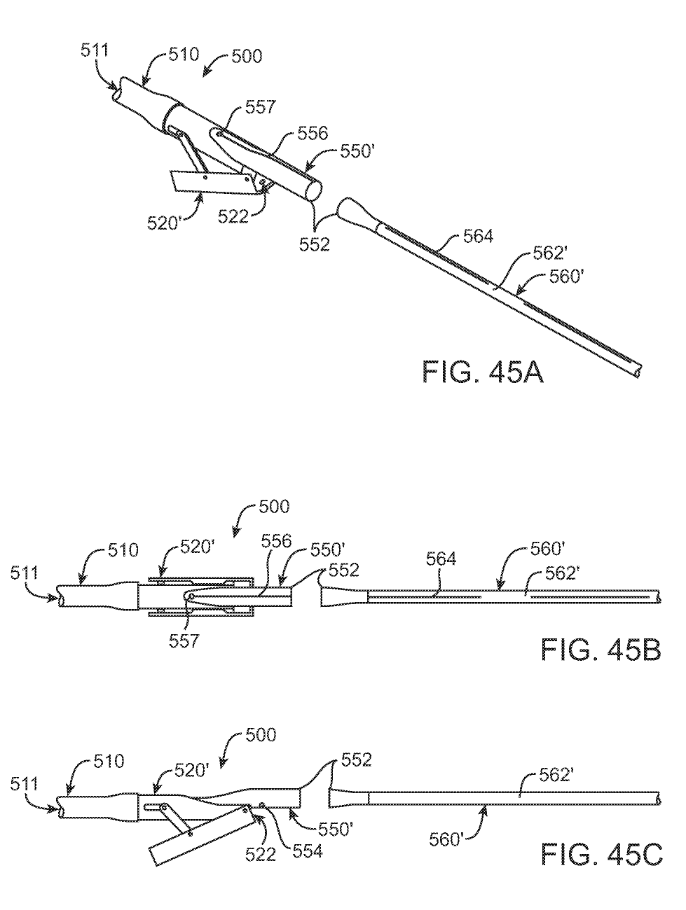

Referring now to FIG. 45, a method of detaching loading device 560′ from liner 550′ is described. Liner 550′ may be torn at perforation 552 to detach the liner from the loading device. Advantageously, detachment perforation 552 allows a medical practitioner to separate the liner from the loading device without requiring additional cutting tools.

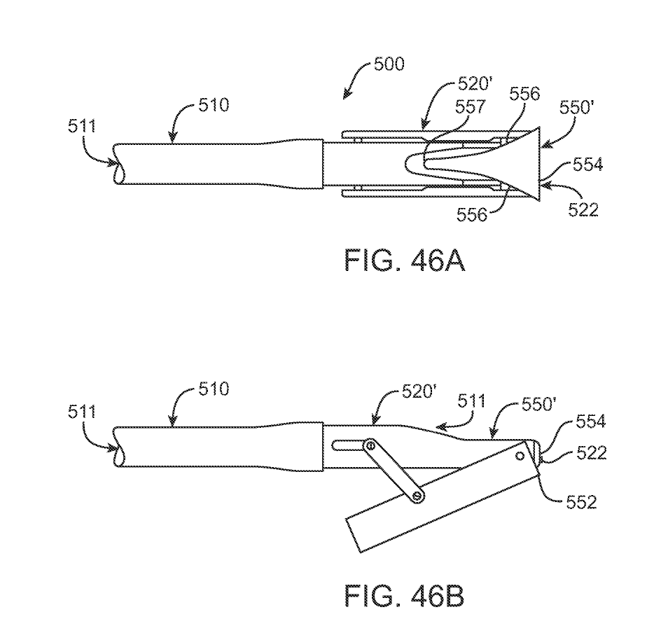

With reference to FIG. 46, an illustrative method of securing liner 550′ to handle 520′ of apparatus 500 is described. As discussed previously, anchoring hole 554 of the liner preferably is aligned with anchor post 522 of the handle, e.g. via alignment indicator 564 of loading device 560′. Once liner 550′ has been separated from loading device 560′ at perforation 552, slit 556 of the liner may be manipulated such that anchoring hole 554 of the liner is distended about anchor post 522 of the handle. In this manner, liner 550′ is reversibly coupled to the handle, thereby reducing a risk of the liner migrating distally within apparatus 500 during advancement of an endoscope or other instrument(s) through lumen 511 of the apparatus. Additional and/or alternative methods and apparatus for securing liner 550′ to handle 520′ will be apparent to those of skill in the art.

In addition to facilitating coupling of the liner to the handle, slit 556 of liner 550′ provides multiple benefits. The slit expands the proximal end of the liner, which facilitates access to lumen 511 of apparatus 500 through the liner. Additionally, the expanded liner acts as a buffer between handle 520′ and an endoscope or other instrument(s) advanced through the handle, thereby protecting the scope/instrument(s) from damage during advancement through the handle. The expanded liner also reduces contact surface area at the mouth of handle 520′, thereby simplifying scope/instrument handling. Furthermore, the expanded liner provides funnel-like access to liner 550′, which facilitates wetting and/or rewetting of the liner. Advantageously, such wetting may be achieved with a standard syringe, as opposed to a specialized irrigator.

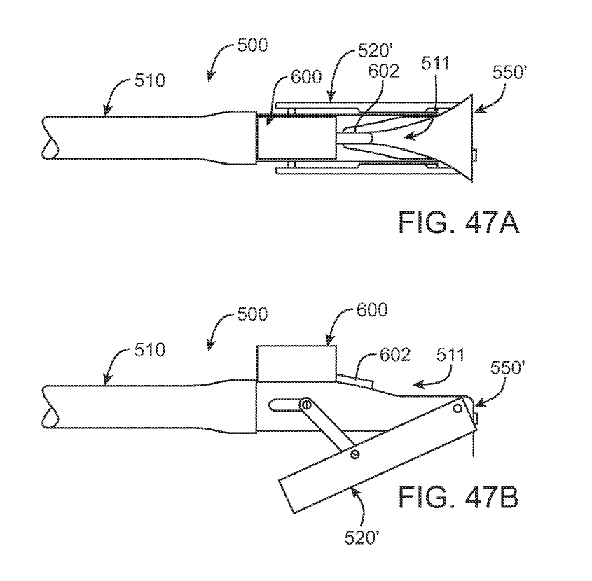

Referring now to FIG. 47, a variation of the apparatus of FIG. 46 is described comprising a fluid reservoir. Fluid reservoir 600 preferably is coupled to handle 520′, but may be positioned at alternative locations. Reservoir 600 illustratively comprises tubing 602, which communicates with lumen 511. Fluids may be deposited within lumen 511 and/or along the shaft of an endoscope or instrument(s) disposed within the lumen, by dispensing fluid from reservoir 600. Exemplary fluids include, but are not limited to, wetting fluids, water, lubricants, hydrophilic fluids and combination thereof.

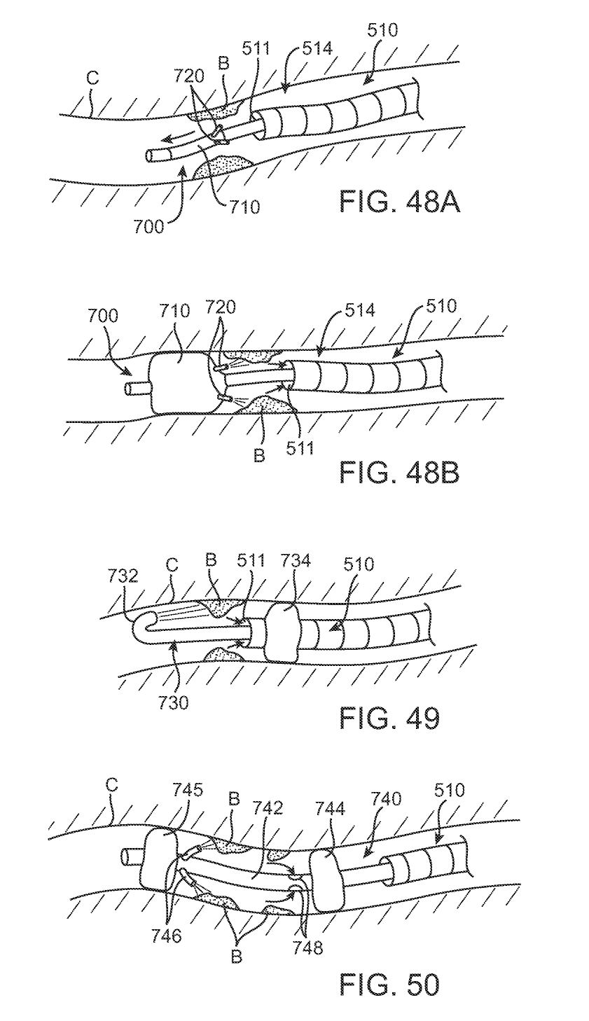

With reference to FIG. 48, apparatus and one method for cleaning a patient's colon in preparation for colonoscopy are described. As mentioned previously, in some patients, colonoscopy cannot be performed due to poor colon cleaning or preparation prior to the procedure. For these and other patients, it may be desirable to clean or prepare the colon as part of the colonoscopy procedure.

As seen in FIG. 48A, distal region 514 of overtube 510 has been positioned within a patient's colon C in the vicinity of one or more localized blockages B, for example, stool and/or fecal matter. Blockage(s) B may obstruct or prevent an examination of the walls of the colon C. Blockage(s) may also inhibit or preclude advancement of overtube 510 and/or of an endoscope, e.g., positioned within lumen 511 of the overtube, through colon C. Thus, colon-cleaning element 700 may be advanced through lumen 511 of the overtube prior to advancement of an endoscope, in order to clean and prepare the colon for further advancement of the overtube and/or of the endoscope. Overtube 510 may be rigidized or transitioned from a flexible to a rigid state, before, during or after advancement of the colon-cleaning element through the overtube.

Colon-cleaning element 700 comprises distal expandable balloon 710 and at least one proximally-oriented fluid jet 720 configured for directing a pressurized stream of fluid at blockage(s) B for loosening, softening, breaking up or otherwise disrupting the blockages and forcing the disrupted material towards and/or out the patient's anus. As seen in FIG. 48A, element 700 may be advanced through lumen 511 of overtube 510 with expandable balloon 710 disposed in a low profile delivery configuration. As seen in FIG. 48B, once the balloon is positioned distal of target blockage(s) B, the balloon is expanded into contact with or adjacent to the wall of colon C. Fluid, e.g., saline, is then delivered through lumens within colon-cleaning element 700 to jet(s) 720 in order to disrupt the blockage(s). Once the blockage(s) have been disrupted, colon-cleaning element 700 may be removed from the patient and colonoscopy may proceed, e.g., as described previously.

Suction optionally may be drawn through lumen 511 or a secondary lumen (not shown) of overtube 510 for aspirating the disrupted material. In such a configuration, the overtube optionally may comprise an inflatable balloon for anchoring the overtube against the wall of the patient's colon and for preventing proximal flow of disrupted blockage material past the overtube, such that the disrupted material is solely evacuated or aspirated through the overtube. Gravity additionally or alternatively may be utilized to evacuate disrupted material.

Referring now to FIG. 49, an alternative colon-cleaning element is described. In FIG. 49, colon-cleaning element 730 having steerable distal tip 732 has been advanced through the lumen of overtube 510 and positioned distal of blockage B. Distal tip 732 has been retroflexed such that it is proximally directed towards the patient's anus. Alternatively, distal tip 732 may be adapted or pre-formed to curve into its retroflexed shape upon distal advancement from overtube 510. Reintroduction of distal tip 732 back into overtube 510 may straighten the curved portion for withdrawal from the patient. Overtube 510 illustratively comprises an optional expandable balloon 734 that reversibly engages the wall of colon C. A jet of fluid is directed through a lumen of the colon-cleaning element in order to disrupt blockage B, which may be aspirated through the overtube. Colon-cleaning element 730 optionally may be rotated to facilitate cleaning of a radial segment or of a full revolution of colon C. Colon-cleaning element 730 optionally may comprise previously described colonoscope 10 have steerable distal tip 11.

With reference to FIG. 50, another alternative colon-cleaning element is described. Colon-cleaning element 740 comprises proximal balloon 744 and distal balloon 745 coupled to flexible shaft 742. At least one fluid jet 746 is disposed along the flexible shaft between the proximal and distal balloons. The at least one fluid jet preferably is positioned in proximity to distal balloon 745. Optionally, at least one suction or aspiration port 748 is also positioned between the proximal and distal balloons, preferably between the fluid jet(s) and proximal balloon 744.

As seen in FIG. 50, in use, colon-cleaning element 740 may be advanced such that blockage(s) B are positioned between the proximal and distal balloons. Once properly positioned, balloons 744 and 745 may be expanded into contact with the wall of colon C. The balloons preferably sealingly engage the wall of the colon. Pressurized fluid is directed through jet(s) 746 to blockage(s) B in order to disrupt the blockages and prepare the colon for colonoscopy. The slurry of disrupted material is aspirated or removed via suction drawn through port(s) 748.

After disrupting and evacuating any blockage, balloons 744 and 745 may be deflated, and colonoscopy may proceed. In one variation, colon-cleaning element 740 comprises a modified endoscope that is subsequently advanced to perform colonoscopy. In another variation, element 740 is removed from the patient, and an endoscope then is advanced within colon C, e.g., through lumen 511 of overtube 510. Colon-cleaning element 740 also may be advanced through the overtube or may prepare the colon prior to placement of the overtube within colon C.

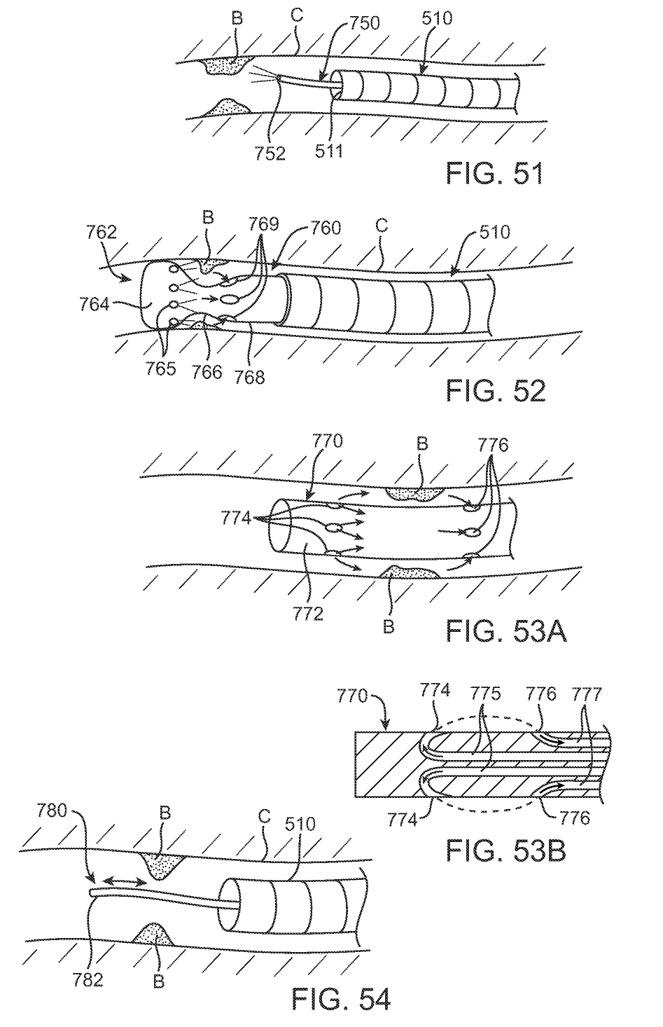

With reference now to FIG. 51, yet another alternative colon-cleaning element is described. Colon-cleaning element 750 comprises distally-directed fluid jet 752 configured for advancement through lumen 511 of overtube 510. Jet 752 disrupts blockage(s) B, and the disrupted material may be aspirated through a lumen of overtube 510.

Referring now to FIG. 52, still another colon-cleaning element is described. Colon-cleaning element 760 comprises distal region 762 having expandable distal end 764, tapered central section 766 and suction proximal end 768. Expandable distal end 764 of distal region 762 comprises fluid jets 765, while suction proximal end 768 of the distal region comprises suction or aspiration ports 769. Expandable distal end 764 may, for example, comprise a self-expanding element or an inflatable balloon.

In use, colon-cleaning element 760 is positioned such that distal end 764 is disposed distal of blockage(s) B and proximal end 768 is disposed proximal. Fluid jets 765 and aspiration ports 769 are directed towards the wall of colon C, such that fluid delivered through jets 765 impinges upon blockage(s) B and then is aspirated through ports 769. After disruption of blockage material, distal end 764 may be collapsed, and colon-cleaning element 760 may be removed through lumen 511 of overtube 510.

With reference to FIG. 53, another colon-cleaning element is described. Colon-cleaning element 770 comprises shaft 772 having distal fluid jets 774 and more proximal aspiration ports 776. As seen in FIG. 53A, fluid jets 774 are proximally directed for disruption of blockage(s) B and for aspiration of the disrupted material through ports 776. Fluid is delivered to the jets through lumens 775, while aspirated material is removed through lumens 777, as seen in the side-section of FIG. 53B.

Referring now to FIG. 54, a colon-cleaning element is described utilizing mechanical, as opposed to fluid, disruption of blockage material. Colon-cleaning element 780 comprises brush 782 having a plurality of bristles 784 for disrupting blockage(s) B. Brush 782 may be advanced through lumen 511 of overtube 510 in a reduced configuration, and bristles 784 may self-expand after being advanced distal of overtube 510. The brush may be reciprocated against blockage(s) B such that bristles 784 mechanically disrupt the blockage material. Brush 782 optionally may be utilized in combination with fluid or a fluid jet to facilitate blockage disruption. Furthermore, suction or aspiration optionally may be provided, e.g., through the lumen of overtube 510.

It will be obvious to one of ordinary skill in the art that, while the above description has emphasized use of apparatus of the present invention in the lower gastro-intestinal tract, and in particular, in performing colonoscopy, the apparatus of the present invention also may be used in the upper gastro-intestinal tract, as well as laparoscopic procedures as a variable rigidity trocar through which a steerable laparoscopic endoscope or tool may be advanced. Apparatus described herein also may be scaled down in size for use in endo-urological procedures. For example, a miniaturized overtube may be advanced, along with a steerable nephroscope, through a patient's ureter into a kidney for access to the kidney's lower pole.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. An apparatus for clearing a colon, comprising:
an overtube having a lumen;
a tensioning mechanism selectively operable to transition the overtube between a flexible state and a rigid state; and
a colon-cleaning element adapted for advancement through the lumen of the overtube.

2. The apparatus of claim 1, wherein the colon-cleaning element is configured to disrupt blockage within a patient's colon in preparation for colonoscopy.

3. The apparatus of claim 2, wherein the colon-cleaning element comprises a fluid jet for disrupting the blockage.

4. The apparatus of claim 3, wherein the fluid jet is proximally-directed.

5. The apparatus of claim 3, wherein the fluid jet is distally-directed.

6. The apparatus of claim 2 further comprising an aspiration element for removing disrupted blockage material from the patient.

7. The apparatus of claim 6, wherein the overtube comprises the aspiration element.

8. The apparatus of claim 6, wherein the colon-cleaning element comprises the aspiration element.

9. The apparatus of claim 1, wherein the colon-cleaning element comprises an engagement element configured to engage a patient's colon.

10. The apparatus of claim 9, wherein the engagement element comprises an expandable balloon configured to sealingly engage the patient's colon.

11. The apparatus of claim 1, wherein the overtube comprises an engagement element configured to engage a patient's colon.

12. The apparatus of claim 11, wherein the engagement element comprises an expandable balloon configured to sealingly engage the patient's colon.

13. The apparatus of claim 2, wherein the colon-cleaning element comprises a mechanical blockage disruption element.

14. The apparatus of claim 13, wherein the mechanical blockage disruption element comprises a brush having a plurality of bristles.

15. A method for cleaning a colon, comprising:
advancing an overtube into a patient's colon to a position proximal of a colonic blockage;
transitioning the overtube from a flexible state to a rigid state;
advancing a colon-cleaning element through a lumen of the overtube; and
disrupting the colonic blockage with the colon-cleaning element.

16. The method of claim 15, wherein disrupting the colonic blockage further comprises directing a jet of pressurized fluid from the colon-cleaning element at the blockage.

17. The method of claim 16, wherein directing a jet of pressurized fluid further comprises directing a proximally-directed jet of pressurized fluid from the colon-cleaning element at the blockage.

18. The method of claim 16, wherein directing a jet of pressurized fluid further comprises directing a distally-directed jet of pressurized fluid from the colon-cleaning element at the blockage.

19. The method of claim 15, wherein disrupting the colonic blockage further comprises mechanically disrupting the colonic blockage with the colon-cleaning element.

20. The method of claim 19, wherein mechanically disrupting the colonic blockage further comprises reciprocating bristles of a brush of the colon-cleaning element against the blockage.

21. The method of claim 15 further comprising aspirating disrupted blockage material from the patient.

22. The method of claim 21, wherein aspirating disrupted blockage material further comprises aspirating the disrupted blockage material through the overtube.

23. The method of claim 21, wherein aspirating disrupted blockage material further comprises aspirating the disrupted blockage material through the colon-cleaning element.

24. The method of claim 15 further comprising reversibly engaging a wall of the patient's colon with the colon-cleaning element.

25. The method of claim 15 further comprising reversibly engaging a wall of the patient's colon with the overtube.

26. The method of claim 15 further comprising:
removing the colon-cleaning element through the overtube; and
advancing an endoscope through the overtube.

27. A method for clearing a colon, comprising:
advancing a rigidizable overtube into a patient's colon to a position proximal of a colonic blockage;
advancing a colon-cleaning element through a lumen of the rigidizable overtube;
disrupting the colonic blockage with the colon-cleaning element;
further advancing the overtube to a bend in the patient's colon;
rigidizing the overtube; and
advancing an endoscope through the overtube to a position distal of the bend.

28. A method for clearing a colon, said method comprising:
advancing a colon-cleaning element to a location proximate a blockage in the colon;
directing a fluid from the element into the blockage to disrupt the blockage; and
aspirating the disrupted blockage material from the colon.

29. A method as in claim 28, wherein the disrupted blockage material is removed through the colon-cleaning element.

30. A method as in claim 29, wherein the colon-cleaning element is advanced through an overtube and the disrupted blockage material is aspirated through the overtube.

31. A method as in claim 30, further comprising occluding the colon at a distal end of the overtube to enhance aspiration.

32. A method as in claim 31, wherein occluding comprises expanding a balloon on the overtube.

33. A method as in claim 29, further comprising at least partially isolating a region around the colon-cleaning element, wherein directing and aspirating are performed in the at least partially isolated region.

34. A method as in claim 33, wherein at least partially isolating comprises expanding at least one balloon.

35. A method as in claim 34, wherein said at least one balloon is on the colon-cleaning element.

36. A method as in claim 33, wherein at least partially isolating comprises expanding at least two balloons.

37. A method as in claim 36, wherein at least one of said at least two balloons is on the colon-cleaning element.

38. A method as in claim 37, wherein both of said at least two balloons are on the colon-cleaning element.

39. A method as in claim 37, wherein a second of said at least two balloons is on an overtube through which the colon-cleaning tube has been placed.
